(19) United States
(12) Patent Application Publication
(10) Pub. No.: US 2023/0346670 A1
(43) Pub. Date: Nov. 2, 2023

(54) Prebiotic Oral Care Compositions and Methods

(71) Applicants: Colgate-Palmolive Company, New York, NY (US); Katholieke Universiteit Leuven, Leuven (BE); Universiteit Gent, Gent (BE)

(72) Inventors: Wim TEUGHELS, Everberg (BE); Tim VERSPECHT, Leuven (BE); Kristel BERNAERTS, Leuven (BE); Marc QUIRYNEN, Leuven (BE); Nico BOON, Gent (BE); Carlo DAEP, Brooklyn, NY (US); James MASTERS, Ringoes, NJ (US)

(21) Appl. No.: 18/029,518

(22) PCT Filed: Sep. 30, 2021
(86) PCT No.: PCT/US2021/052928

§ 371 Date: Mar. 30, 2023

Related U.S. Application Data
(60) Provisional application No. 63/086,846, filed on Oct. 2, 2020.

(51) Int. Cl. A61Q 11/00; A61K 8/60

(52) U.S. Cl. CPC A61K 8/60; A61Q 11/00; A61K 2800/70

(57) ABSTRACT

The disclosure relates to methods of enhancing beneficial oral bacteria and decreasing harmful oral bacteria comprising administering oral care compositions comprising a saccharide prebiotic, e.g., selected from hemolysin, N-acetyl-D-glucosamine, α-D-lactose, D-(+)-trehalose or D-(+)-raffinose and mixtures thereof; and oral care compositions for use in such methods. The disclosure also relates to methods of using prebiotic oral care compositions, methods of screening, and methods of manufacture.

### FIELD

The field relates to methods of enhancing beneficial oral bacteria and decreasing harmful oral bacteria comprising administering oral care compositions comprising a saccharide prebiotic, e.g., a saccharide selected from N-acetyl-D-glucosamine, α-D-lactose, D-(+)-trehalose or D-(+)-raffinose; and oral care compositions for use in such methods. The field also relates to methods of using prebiotic oral care compositions, methods of screening, and methods of manufacture.

### BACKGROUND

Different types of sugar are present in a given diet and may come into contact with plaque during eating. The breakdown of sugars is an important step that influences the plaque environment. Sugar metabolism requires specific enzymes. The genetic disposition and expression of pathway dictates which strains are able to grow on which type of sugars.

The occurrence of high amount of certain sugar may provide a selection advantage to certain species over others, simply due to the fact that they are able to grow on the metabolite but also due to effects that influence the environment such as acid production, bacteriocins, and/or breakdown products that may be metabolized by further species.

When there is an increase in the intake of certain fermentable carbohydrates, this may cause pH to drop in a user’s oral cavity. Not only does the acid damage the teeth, but the acidic environment causes a shift toward a more aciduric and acidogenic bacteria, and certain cariogenic bacteria, which are typically found in relatively small amounts, may actually increase in number and size. Ultimately, this can lead to dental caries. Some species of oral pathogenic bacteria (e.g. Porphyromonas gingivalis, Tannerella forsythia and Aggregatibacter actinomycetemcomitans) have been implicated in the development of periodontal diseases such as periodontitis, gingivitis, necrotizing periodontitis, necrotizing gingivitis and peri-implantitis. Certain species of oral pathogenic bacteria have been implicated in tooth decay (e.g. Streptococcus mutans). Current strategies to address these problems include the use of oral care products containing broad-spectrum antibacterial agents. Such a product, however, can inhibit or kill bacteria irrespective of whether the bacteria are beneficial or detrimental. Moreover, pathogens may evolve to develop resistance to antimicrobial agents. Accordingly, alternative methods of prophylaxis and treatment are needed.

“Probiotics” are microorganisms that provide health benefits when consumed. “Prebiotics” are ingestible ingredients that allow specific changes, both in the composition and/or activity in the gastrointestinal microflora that confer benefits upon the host well-being and health. While prebiotics may be generally known for influencing the composition of the gastrointestinal microflora, there has been relatively little attention directed to using a similar prebiotic strategy to encourage beneficial oral bacteria. Rather than trying to stimulate beneficial bacteria in the mouth, the emphasis has been on avoiding and promptly removing compounds, like sucrose, that encourage harmful oral bacteria as well as using antibacterial agents to reduce oral plaque.

The use of prebiotics for maintaining and/or restoring the health-associated homeostasis of the oral microbiota and modulation of the host response is being investigated more. However, there is currently a need for the stimulation of beneficial/commensal bacteria through potentially prebiotic substrates, resulting in more host-compatible biofilms that harbor lower amounts of pathogens, show decreased virulence and have less inflammatory potential. It would be desirable to identify new substrates, for example, which can stimulate the beneficial/commensal oral bacteria in terms of growth and/or metabolism and by consequence inhibit pathogenic oral bacteria, decrease virulence gene expression and reduce the inflammatory response of oral keratinocytes exposed to substrate-treated biofilms.

### SUMMARY OF THE INVENTION

The invention contemplates that certain in vitro multi-species oral biofilms can surprisingly be modulated by stimulating certain beneficial/commensal bacteria with potentially prebiotic substrates, e.g., saccharide prebiotics. This stimulation can result in more host-compatible biofilms that comprise lower amounts of pathogens, demonstrate decreased virulence and have less inflammatory potential as measured by certain inflammatory biomarkers.

In one aspect, the substrates (e.g., saccharide prebiotics), can stimulate certain beneficial/commensal oral bacteria in terms of growth and/or metabolism. In yet another aspect, by stimulating certain beneficial/commensal oral bacteria also results in the inhibition of certain pathogenic oral bacteria, decrease virulence gene expression and reduce the inflammatory response of oral keratinocytes exposed to multi-species oral biofilms that are treated with these substrates.

The inventors have surprisingly found that four new potentially prebiotic substrates exhibit certain concentration-dependent modulatory effects that cause in vitro multi-species oral biofilms to become more host-compatible. The inventors are successfully able to investigate the effects of potentially prebiotic substrates on composition, metabolic activity, virulence gene expression and inflammatory potential of an in vitro, complex 14-species oral biofilm.

Oral care compositions comprising a saccharide prebiotic identified in this manner, e.g., saccharide prebiotics selected from N-acetyl-D-glucosamine, α-D-lactose, D-(+)-trehalose or D-(+)-raffinose, and combinations thereof, are found to increase the growth of certain beneficial/commensal bacteria in the oral microbiota. Such beneficial bacteria can include, e.g., A. naeslundii; A. viscosus; S. gordonii: S. mitis; S. oralis; S. salivarius; S. sanguinis; V. parvula. These selected saccharide prebiotics that encourage the growth of beneficial bacteria also negatively affect the growth of certain pathogenic strains of bacteria. These pathogenic strains can include, e.g.: A. actinomycetemcomitans; F. nucleatum; P. gingivalis; P. intermedia; S. sobrinus.

The present invention contemplates that selective stimulation of beneficial bacteria provides a valid preventive approach for oral health. Without being bound by any theory, it is thought that since bacteria need certain substrates in order to grow, one can obtain certain microbiological shifts in the bacterial environment by selectively encouraging the growth of an individual’s beneficial endogenous bacterial population by providing them with appropriate substrates. For example, without being bound by theory, select substrates are preferentially utilized by certain microorganisms. By selecting the appropriate substrate, it is possible encourage the growth of certain microorganisms (e.g., beneficial endogenous bacterial strains) while also directly or indirectly suppressing the growth of select other microorganisms (endogenous pathogenic bacterial strains).

In one aspect, the invention relates to a novel prebiotic approach that selectively promotes the growth of beneficial endogenous bacteria but not the growth of harmful bacteria by using an oral care composition comprising a prebiotically effective amount of a saccharide prebiotic, e.g., a saccharide prebiotic selected from N-acetyl-D-glucosamine, α-D-lactose, D-(+)-trehalose or D-(+)-raffinose, and combinations thereof. For example, this may include use of compositions which promote the growth of at least one of the above-listed beneficial bacteria while not simultaneously promoting growth of any of the above-listed harmful bacteria.

An oral care composition (Composition 1) useful in the methods of the present invention is an oral care composition comprising an effective amount of at least one saccharide prebiotic, e.g., a saccharide prebiotic selected N-acetyl-D-glucosamine, α-D-lactose, D-(+)-trehalose or D-(+)-raffinose and mixtures thereof, e.g., in an amount effective to promote the growth of beneficial endogenous bacteria in the oral cavity and inhibit pathogenic oral bacteria (e.g., decrease virulence gene expression and reduce the inflammatory response of oral keratinocytes) . For example, in various aspects the oral care compositions useful in the methods of the present invention include:
1.1 Composition 1, wherein the saccharide prebiotic is a mono-, di- or trisaccharide, e.g., selected from N-acetyl-D-glucosamine, α-D-lactose, D-(+)-trehalose or D-(+)-raffinose, and mixtures thereof.
1.2 Composition 1, wherein the saccharide prebiotic is a monosaccharide e.g., N-acetyl-D-glucosamine.
1.3 Composition 1, wherein the saccharide prebiotic is a disaccharide, e.g., α-D-lactose or D-(+)-trehalose.
1.4 Composition 1, wherein the saccharide prebiotic is an trisaccharide, e.g., D-(+)-raffinose.
1.5 Composition 1 wherein the saccharide prebiotic is N-acetyl-D-glucosamine.
1.6 Composition 1 wherein the saccharide prebiotic is D-lactose.
1.7 Composition 1 wherein the saccharide prebiotic is D-(+)-trehalose.
1.8 Any foregoing composition wherein the amount of saccharide prebiotic is at least 0.1%, e.g., 0.1% to 5%, e.g., about 0.5%, 1% or 2% by weight of the composition.
1.9 Any foregoing composition wherein the amount of saccharide prebiotic is from 0.5 µmol/L to 10 mmol/L, from 0.5 µmol/L to 5 mmol/L, from 1 µmol/L to 5 mmol/L, from 5 µmol/L to 10 mmol/L, from 0.75 µmol/L to 1.5 mmol/L, about 0.75 mmol/L, about 1 mmol/L, about 1.5 mmol/L or about 1.75 mmol/L.
1.10 Any foregoing composition wherein the saccharide prebiotic is not derived from a plant extract.
1.11 Any foregoing composition wherein the composition promotes the growth or expression in the oral cavity of one or more beneficial or commensal endogenous bacterial species, wherein said species are one or more selected from the group consisting of: A. naeslundii; A. viscosus; S. gordonii: S. mitis; S. oralis; S. salivarius; S. sanguinis; V. parvula and combinations thereof.
1.12 Any foregoing composition, wherein the composition negatively affects the growth or expression in the oral cavity of one or more pathogenic bacterial species, wherein said species are one or more selected from the group consisting of: A. actinomycetemcomitans; F. nucleatum; P. gingivalis; P. intermedia; S. Sobrinus and combinations thereof.
1.13 The composition of 1.12, wherein the composition negatively affects the growth in the oral cavity of one or more pathogenic bacterial species, wherein said species are one or more selected from the group consisting of A. actinomycetemcomitans; F. nucleatum; P. gingivalis; P. intermedia and combinations thereof.
1.14 Any foregoing composition wherein the composition further comprises at least one species of bacteria that has beneficial effects on oral health.
1.15 Composition 1.14 wherein the species of bacteria that has beneficial effects on oral health is selected from A. naeslundii; A. viscosus; S. gordonii: S. mitis; S. oralis; S. salivarius; S. sanguinis; V. parvula and combinations thereof.
1.16 Any of the preceding compositions further comprising an anti-calculus agent.
1.17 Any of the preceding compositions further comprising an anti-calculus agent which is a polyphosphate, e.g., pyrophosphate, tripolyphosphate, or hexametaphosphate, e.g., in sodium salt form.
1.18 Any of the preceding compositions comprising at least one surfactant selected from sodium lauryl sulfate, cocamidopropyl betaine, and combinations thereof (e.g., from 0.5-5% by wt of the composition).
1.19 Any of the preceding compositions comprising an anionic surfactant, e.g., selected from sodium lauryl sulfate, sodium laureth sulfate, and mixtures thereof.
1.20 Any of the preceding compositions comprising sodium lauryl sulfate, in an amount from 0.5-5% by wt of the composition.
1.21 Any of the preceding compositions comprising at least one humectant (e.g., from 1% - 50% by wt. of the composition).
1.22 Any of the preceding compositions comprising at least one humectant selected from glycerin, sorbitol and combinations thereof.
1.23 Any of the preceding compositions comprising at least one polymer.
1.24 Any of the preceding compositions comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.25 Any of the preceding compositions comprising one or more abrasives, e.g., silica, calcium carbonate, or calcium phosphate abrasives.
1.26 Any of the preceding compositions comprising gum strips or fragments.
1.27 Any of the preceding compositions comprising flavoring, fragrance and/or coloring.
1.28 Any composition obtained or obtainable by combining the ingredients as set forth in any of the preceding compositions.
1.29 Any of the preceding oral care compositions, wherein the composition is a mouthwash, toothpaste, tooth gel, tooth powder, non-abrasive gel, mousse, foam, mouth spray, lozenge, oral tablet, or dental implement.
1.30 Any of the preceding compositions wherein the composition is a toothpaste or a mouthwash.
1.31 Any of the preceding compositions wherein the composition is a toothpaste optionally further comprising one or more of one or more of water, abrasives, surfactants, foaming agents, vitamins, polymers, enzymes, humectants, thickeners, preservatives, flavorings, colorings and/or combinations thereof.
1.32 Any preceding composition, wherein the composition is a toothpaste further comprising water, abrasive, surfactant, humectant, thickener, and flavoring.
1.33 Any preceding composition wherein the composition is a toothpaste obtained or obtainable by a method of mixing with a toothpaste base, e.g., a toothpaste base comprising one or more of one or more of water, abrasives, surfactants, foaming agents, vitamins, polymers, enzymes, humectants, thickeners, antimicrobial agents, preservatives, flavorings, colorings and/or combinations thereof.
1.34 Any preceding composition for use in selectively promoting, in an oral cavity: growth, metabolic activity or colonization of bacteria that have beneficial effects on oral health, relative to growth, metabolic activity or colonization of pathogenic oral bacteria.
1.35 Composition 1.34 wherein the bacteria that have beneficial effects on oral health are selected from A. naeslundii; A. viscosus; S. gordonii: S. mitis; S. oralis; S. salivarius; S. sanguinis; V. parvula and combinations thereof.
1.36 Any of compositions 1.34 to 1.35 wherein the pathogenic oral bacteria are selected from A. actinomycetemcomitans; F. nucleatum; P. gingivalis; P. intermedia; S. mutans; S. Sobrinus and combinations thereof.
1.37 Composition 1.36, wherein the pathogenic oral bacteria are selected from A. actinomycetemcomitans; F. nucleatum; P. gingivalis; P. intermedia, and combinations thereof.
1.38 Any of compositions 1.33 to 1.37, wherein the composition selectively promotes growth, metabolic activity or colonization of bacteria that have beneficial effects on oral health, relative to growth, metabolic activity or colonization of pathogenic oral bacteria, after 24 hours incubation with the bacteria that have beneficial effects on oral health and the pathogenic oral bacteria.
1.39 Any of compositions 1.33 to 1.37, wherein the composition selectively promotes growth, metabolic activity or colonization of bacteria that have beneficial effects on oral health, relative to growth, metabolic activity or colonization of pathogenic oral bacteria, after 48 hours incubation with the bacteria that have beneficial effects on oral health and the pathogenic oral bacteria.
1.40 Any preceding composition for use in selectively promoting, in an oral cavity, biofilm formation by bacteria that have beneficial effects on oral health, relative to biofilm formation by pathogenic oral bacteria.
1.41 Composition 1.40 wherein the bacteria that have beneficial effects on oral health are selected from A. naeslundii; A. viscosus; S. gordonii: S. mitis; S. oralis; S. salivarius; S. sanguinis; V. parvula, and combinations thereof.
1.42 Any of compositions 1.40 or 1.41 wherein the pathogenic oral bacteria are selected from A. actinomycetemcomitans; F. nucleatum; P. gingivalis; P. intermedia; S. mutans; S. Sobrinus, and combinations thereof.
1.43 Composition 1.42, wherein the pathogenic bacteria is selected from: A. actinomycetemcomitans; F. nucleatum; P. gingivalis; P. intermedia, and combinations thereof.
1.44 Any of the preceding compositions, wherein the prebiotic substrate can decrease virulence gene expression of one or more of the following genes in A. actinomycetemcomitans: flp, aae, apaH, cdtB, emaA, ltxA, omp100, omp29, orf859, pgA, vapA.
1.45 Any of the preceding compositions, wherein the prebiotic substrate can decrease virulence gene expression of one or more of the following genes in P. gingivalis: fim A, kgp, partC, rgpA and combinations thereof.
1.46 Any of the preceding compositions, wherein the prebiotic substrate can decrease virulence gene expression of one or more of the following genes in P. intermedia: adpc, clpB, DnaK, DnaJ, ECF, GroES, HtpG, KpsD, inpA, phg and combinations thereof.
1.47 Any of the preceding compositions, wherein the prebiotic substrate can decrease virulence gene expression of one or more of the following genes in F. nucleatum: butyrate-acetoacetate CoA-transferase, ABC transporter permease, transposase, hemolysin, hemin receptor, ompA, EF-G and combinations thereof.
1.48 Any of the preceding compositions, wherein the prebiotic substrate can decrease the absolute number (e.g., measured via qPCR) of one or more pathogenic bacteria selected from: A. actinomycetemcomitans; F. nucleatum; P. gingivalis; P. intermedia, and combinations thereof.
1.49 Any of the preceding compositions, wherein treatment with a saccharide prebiotic (e.g., N-acetyl-D-glucosamine, α-D-lactose, D-(+)-trehalose or D-(+)-raffinose, and mixtures thereof) results in an oral microbiota with greater than 60% beneficial/commensal bacteria (e.g., from 60% - 98% beneficial/commensal bacteria (Geq/mL)) (e.g., from 90% - 98% beneficial/commensal bacteria (Geq/mL).
1.50 Any of the preceding compositions, wherein treatment with a saccharide prebiotic (e.g., N-acetyl-D-glucosamine, α-D-lactose, D-(+)-trehalose or D-(+)-raffinose, and mixtures thereof) results in an oral microbiota with less than 40% periodontal pathogenic bacterial species (e.g., from 1.25% - 39% periodontal pathogenic bacterial species (Geq/mL)) (e.g., from 25% - 39% periodontal pathogenic bacterial species (Geq/mL)) (e.g., from 1.25% - 2.5% periodontal pathogenic bacterial species (Geq/mL)) .
1.51 Any of the preceding compositions, wherein treatment with a saccharide prebiotic (e.g., N-acetyl-D-glucosamine, α-D-lactose, D-(+)-trehalose or D-(+)-raffinose, and mixtures thereof) can decrease the inflammatory response in oral keratinocytes.
1.52 The composition of 1.51, wherein the saccharide prebiotic can decrease the gene expression of inflammatory biomarker in oral keratinocytes, wherein the biomarker is selected from: IL-1β, IL-6, IL-8, MMP-8, TNF-α, and combinations thereof.
1.53 Any of the preceding compositions, wherein the saccharide prebiotic can decrease organic acid production in biofilm.
1.54 Composition 1.53, wherein the organic acid is selected from lactate, formate, acetate, propionate, butyrate, and combinations thereof.

For example, the invention provides in one embodiment, an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq. for use in selectively promoting, in an oral cavity: growth, metabolic activity or colonization of bacteria that have beneficial effects on oral health, relative to growth, metabolic activity or colonization of pathogenic oral bacteria.

For example, the invention provides in another embodiment, an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq. for use in selectively promoting, in an oral cavity, biofilm formation by bacteria that have beneficial effects on oral health, relative to biofilm formation by pathogenic oral bacteria.

For example, the invention provides in another embodiment, an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq. for use in maintaining and/or re-establishing a healthy oral microbiota.

For example, the invention provides in another embodiment, an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq. for use in treating or preventing one or more of gingivitis, periodontitis, peri-implantitis, peri-implant mucositis, necrotizing gingivitis, necrotizing periodontitis, systemic health disorders and caries.

Further provided is a method for prophylaxis or reduction of tooth decay, caries and/or gum disease, comprising contacting the oral cavity with a composition comprising an effective amount of at least one saccharide prebiotic (e.g., any of Composition 1, et seq), e.g., a saccharide prebiotic selected from N-acetyl-D-glucosamine, α-D-lactose, D-(+)-trehalose or D-(+)-raffinose and mixtures thereof, e.g., any of Compositions 1, et seq., e.g by brushing, e.g. on a regular basis over a sufficient period of time to enhance the growth of beneficial bacteria in the oral cavity.

Further provided is a method for increasing the amount of beneficial endogenous bacteria in the oral cavity of a subject in need thereof comprising administering to a subject an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq., e.g., wherein the amount of saccharide prebiotic in the composition promotes the growth of beneficial endogenous bacteria, e.g., wherein the beneficial endogenous bacteria are one or more species selected from the group consisting of A. naeslundii; A. viscosus; S. gordonii: S. mitis; S. oralis; S. salivarius; S. sanguinis; V. parvula and combinations thereof.

Further provided is a method of selectively promoting, in an oral cavity of a subject: growth, metabolic activity or colonization of bacteria that have beneficial effects on oral health, relative to growth, metabolic activity or colonization of pathogenic oral bacteria; the method comprising contacting the oral cavity with an oral care composition an effective amount of at least one saccharide prebiotic, e.g., a saccharide prebiotic selected N-acetyl-D-glucosamine, α-D-lactose, D-(+)-trehalose or D-(+)-raffinose and mixtures thereof, e.g., any of Composition 1, et seq.

Further provided is a method of selectively promoting, in an oral cavity of a subject, biofilm formation by bacteria that have beneficial effects on oral health, relative to biofilm formation by pathogenic oral bacteria; the method comprising contacting the oral cavity with an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Composition 1, et seq.

Further provided is a method for decreasing the amount of pathological endogenous bacteria in the oral cavity of a subject in need thereof comprising administering to a subject an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Composition 1, et seq., e.g., wherein the amount of the saccharide prebiotic in the composition inhibits the growth of pathological endogenous bacteria, e.g., wherein the pathological endogenous bacteria are one or more species selected from the group consisting of: A. actinomycetemcomitans; F. nucleatum; P. gingivalis; P. intermedia; S. Sobrinus and combinations thereof.

Further provided is a method of maintaining and/or re-establishing a healthy oral microbiota in a subject, the method comprising contacting an oral cavity of the subject with an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Compositions 1, et seq.

Further provided is a method of preventing or mitigating or treating one or more of gingivitis, periodontitis, peri-implantitis, peri-implant mucositis, necrotizing gingivitis, necrotizing periodontitis, systemic health disorders and caries in a subject, by selectively promoting, in an oral cavity of a subject: growth, metabolic activity or colonization of bacteria that have beneficial effects on oral health, relative to growth, metabolic activity or colonization of pathogenic oral bacteria, the method comprising contacting an oral cavity of the subject with an oral care composition comprising an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Composition 1, et seq.

Further provided is a use of a saccharide prebiotic, e.g., a saccharide prebiotic selected from N-acetyl-D-glucosamine, α-D-lactose, D-(+)-trehalose or D-(+)-raffinose and mixtures thereof, e.g., in any of Compositions 1, et seq., for prophylaxis or reduction of tooth decay, caries and/or gum disease, or to enhance the growth of beneficial bacteria in the oral cavity, e.g., by contacting the dental surface with a an effective amount of at least one saccharide prebiotic, e.g., a saccharide prebiotic selected from N-acetyl-D-glucosamine, α-D-lactose, D-(+)-trehalose or D-(+)-raffinose and mixtures thereof, e.g., any of Composition 1, et seq.

Further provided is a use of a saccharide prebiotic, e.g., a saccharide prebiotic selected from N-acetyl-D-glucosamine, α-D-lactose, D-(+)-trehalose or D-(+)-raffinose and mixtures thereof, in the manufacture of an oral care composition, e.g., any of Compositions 1, et seq., for prophylaxis or reduction of tooth decay, caries and/or gum disease, or to enhance the growth of beneficial bacteria in the oral cavity.

In still another aspect, the invention relates to the use of a saccharide prebiotic, e.g., a saccharide prebiotic selected from N-acetyl-D-glucosamine, α-D-lactose, D-(+)-trehalose or D-(+)-raffinose and mixtures thereof, in the manufacture of an oral care product, e.g., any of Compositions 1, et seq., to promote growth of beneficial endogenous bacteria, but not the growth of harmful bacteria.

Further provided is use, in an oral care composition (e.g. any of Composition 1, et seq.) of a saccharide prebiotic, e.g., a saccharide prebiotic selected from N-acetyl-D-glucosamine, α-D-lactose, D- (+)-trehalose or D-(+)-raffinose and mixtures thereof, to: (a) selectively promote growth, metabolic activity or colonization of bacteria that have beneficial effects on oral health, relative to growth, metabolic activity or colonization of pathogenic oral bacteria; (b) selectively promote biofilm formation by bacteria that have beneficial effects on oral health, relative to biofilm formation by pathogenic oral bacteria; (c) maintain and/or re-establish a healthy oral microbiota in a subject; or(d) prevent one or more of gingivitis, periodontitis, peri-implantitis, peri-implant mucositis, necrotizing gingivitis, necrotizing periodontitis and caries in a subject.

In another aspect, the invention relates to methods of screening for compounds that promote the growth of beneficial oral bacteria, wherein screening steps include:
Determining the ability of a first compound (e.g., test compound) to promote the growth of beneficial oral bacteria, while simultaneously negatively affects the growth of pathogenic oral bacteria, e.g., comparing growth of at least one species of beneficial oral bacteria and at least one species of pathogenic oral bacteria, e.g., wherein effect of the first compound on growth is measured by optical density or biofilm formation following at least 24 hours culture in the presence and absence of the first compound;
Optionally determining the ability of a second compound (e.g., control compound) to promote the growth of beneficial bacteria, while simultaneously negatively affecting the growth of pathogenic oral bacteria;
Optionally comparing the profile of the first compound with the profile of the second compound;
Selecting a test compound for further testing based upon its ability to promote the growth of beneficial oral bacteria and inhibit the growth of pathogenic oral bacteria, e.g., in comparison to the control compound.
For example, the control compound in the foregoing method of screening may be a saccharide prebiotic, e.g., a saccharide prebiotic selected from N-acetyl-D-glucosamine, α-D-lactose, D-(+)-trehalose or D-(+)-raffinose and mixtures thereof, e.g., any ofCompositions 1, et seq. In some embodiments, the beneficial oral bacteria are one or more species selected from the group consisting of A. naeslundii; A. viscosus; S. gordonii: S. mitis; S. oralis; S. salivarius; S. sanguinis; V. parvula. In some embodiments, the pathogenic oral bacteria are one or more species selected from the group consisting of: A. actinomycetemcomitans; F. nucleatum; P. gingivalis; P. intermedia; S. Sobrinus. The invention further provides the use of a compound identified in such a screening method in any of the foregoing methods and uses.

### DETAILED DESCRIPTION

Unless otherwise indicated, the terms “%” or “percent” when used in connection with an ingredient of the oral care compositions of the invention is intended to refer to the percent by weight of the indicated ingredient relative to the total weight of the oral care composition.

As used herein, “cleaning” generally refers to the removal of contaminants, dirt, impurities, and/or extraneous matter on a target surface. For example, in the context of oral surfaces, where the surface is tooth enamel, the cleaning may remove at least some of a film or stain, such as plaque biofilm, pellicle or tartar.

The terms “indigenous” and “endogenous” are used interchangeably throughout this disclosure.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice. As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

Saccharide prebiotics for use in the present invention are sugars or sugar derivatives, e.g., amide derivatives, amino sugars or sugar alcohols, for example mono-, di- or tri-saccharides (including amino-saccharides and sugar alcohols) which are orally acceptable (i.e., non-toxic at relevant concentrations in an oral care formulation) and which promote the growth of beneficial oral bacteria, while simultaneously negatively affecting the growth of pathogenic oral bacteria. In particular embodiments, the saccharide prebiotic is selected from N-acetyl-D-glucosamine, α-D-lactose, D-(+)-trehalose or D-(+)-raffinose and mixtures thereof and mixtures thereof.

“N-acetyl-D-glucosamine” is a monosaccharide which is an amide derivative of the monosaccharide glucose, and it is also known as N-Acetyl-D-glucosamine, GlcNAc, NAG, and NADG. Its IUPAC name is β-D-(Acetylamino)-Δdeoxy-glucopyranose.

“α-D-lactose” is a disaccharide derived from the condensation of galactose and glucose. Its IUPAC name is β-D-galactopyranosyl-(1→4)-D-glucose. The glucose can be in either the α-pyranose form or the β-pyranose form, whereas the galactose can only have the β-pyranose form: hence α-lactose and β-lactose refer to the anomeric form of the glucopyranose ring alone. “α-D-lactose” is used interchangeably with “D-lactose” herein.

“D-(+)-trehalose” is a sugar consisting of two molecules of glucose. It is also known as mycose or tremalose, and its IUPAC name is: (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol. Trehalose is a nonreducing sugar formed from two glucose units joined by a 1-1 alpha bond, giving it the name α-D-glucopyranosyl-(1→1)-α-D-glucopyranoside. “D-(+)-trehalose” is used interchangeably with “D-trehalose” herein.

“D-(+)-raffinose” is a trisaccharide composed of galactose, glucose, and fructose, and its IUPAC name is: (2R,3R,4S,5S,6R)-Δ[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol. “D-(+)-raffinose” is used interchangeably with “D-raffinose” herein.

In some aspects, the oral care compositions disclosed herein, e.g., any of Composition 1, et seq., comprise water. Water employed in the preparation of the oral care compositions disclosed herein, e.g., any of Composition 1, et seq., should be deionized and free of organic impurities. Water may make up the balance of the oral care composition. In some embodiments, the oral care compositions disclosed herein, e.g., any of Composition 1, et seq., comprise 0 to 90 weight % water, e.g., 0.1 to 90 weight % water, e.g., 1 to 80 weight % water, e.g., 2 to 70 weight % water, 5 to 60 weight % water, e.g., 5 to 50 weight % water, e.g., 20 to 60 weight % water, e.g., 10 to 40 weight % water. This amount of water includes the free water that is added plus that amount which is introduced with other components of the oral care composition, such as with sorbitol.

A thickener provides a desirable consistency and/or stabilizes and/or enhances performance (e.g., provides desirable active release characteristics upon use) of the oral care composition. In some embodiments, the oral care compositions disclosed herein, e.g., any of Composition 1, et seq., comprise from 0.01 to 15 weight % of a thickener, 0.1 to 15 weight % of a thickener, e.g., 0.1 to 10 weight % of a thickener, e.g., 0.1 to 5 weight % of a thickener, e.g., 0.5 to 10 weight % of a thickener, e.g., 0.5 to 5 weight % of at a thickener, e.g., 1 to 4 weight % of a thickener, e.g., 2 to 5 weight % of a thickener, e.g., 2 to 4 weight % of a thickener, e.g., 3 to 4 weight % of a thickener. Higher weight percentages may be used for chewing gums, lozenges and breath mints, sachets, non-abrasive gels and subgingival gels. Thickeners that may be used in the oral care compositions disclosed herein, e.g., any of Composition 1, et seq., include, for example, carboxyvinyl polymers, carrageenan (also known as carrageenan gum), hydroxyethyl cellulose (HEC), natural and synthetic clays (e.g., Veegum and laponite), water soluble salts of cellulose ethers (e.g., sodium carboxymethylcellulose (CMC) and sodium carboxymethyl hydroxyethyl cellulose), natural gums (e.g., gum karaya, xanthan gum, gum arabic, and gum tragacanth), colloidal magnesium aluminum silicate, silica (e.g., finely divided silica), cross-linked poly(vinyl)pyrrolidone, carbowaxes, fatty acids and salts thereof (e.g., stearic acid and palmitic acid), fatty alcohols (e.g., stearyl alcohol), and mixtures thereof. In some embodiments, a mixture of thickening silica and carrageenan gum is used as the thickener in the oral care compositions disclosed herein, e.g., any of Composition 1, et seq. In some embodiments, the oral care compositions disclosed herein, e.g., any of Composition 1, et seq., comprise from 0.01 to 15 weight % of thickening silica and carrageenan gum, 0.1 to 15 weight % of thickening silica and carrageenan gum, e.g., 0.1 to 10 weight % of thickening silica and carrageenan gum, e.g., 0.1 to 5 weight % of thickening silica and carrageenan gum, e.g., 0.5 to 10 weight % of thickening silica and carrageenan gum, e.g., 0.5 to 5 weight % of thickening silica and carrageenan gum, e.g., 1 to 4 weight % of thickening silica and carrageenan gum, e.g., 2 to 5 weight % of thickening silica and carrageenan gum, e.g., 2 to 4 weight % of thickening silica and carrageenan gum, e.g., 3 to 4 weight % of thickening silica and carrageenan gum.

A buffer adjusts the pH of oral care compositions, for example, to a range of about pH 4.0 to about pH 9.5. In some embodiments, the oral care compositions disclosed herein, e.g., any of Composition 1, et seq., comprise from 0.1 to 10 weight % of a buffer, 0.5 to 10 weight % of a buffer, e.g., 0.5 to 5 weight % of a buffer, e.g., 0.5 to 4 weight % of a buffer, e.g., 0.5 to 3 weight % of a buffer, e.g., 0.5 to 2 weight % of a buffer, e.g., 1 to 2 weight % of a buffer. Buffers that may be used in the oral care compositions disclosed herein, e.g., any of Composition 1, et seq., include, for example, sodium bicarbonate, sodium phosphate {e.g., monosodium phosphate (NaH2PO4), disodium phosphate (Na2HPO4), trisodium phosphate (Na3PO4)}, sodium hydroxide, sodium carbonate, sodium acid pyrophosphate, citric acid, sodium citrate, and mixtures thereof. In some embodiments, sodium hydroxide is used as the buffer in the oral care compositions disclosed herein, e.g., any of Composition 1, et seq. In some embodiments, the oral care compositions disclosed herein, e.g., any of Composition 1, et seq., comprise from 0.1 to 10 weight % of sodium hydroxide, e.g., 0.5 to 10 weight % of sodium hydroxide, e.g., 0.5 to 5 weight % of sodium hydroxide, e.g., 0.5 to 4 weight % of sodium hydroxide, e.g., 0.5 to 3 weight % of sodium hydroxide, e.g., 0.5 to 2 weight % of sodium hydroxide, e.g., 1 to 2 weight % of sodium hydroxide.

A humectant keeps oral care compositions from hardening upon exposure to air. Certain humectants can also impart desirable sweetness or flavor to oral care compositions. In some embodiments, the oral care compositions disclosed herein, e.g., any of Composition 1, et seq., comprise, on a pure humectant basis, from 0 to 70 weight % of a humectant, e.g., 10 to 70 weight % of a humectant, e.g., 10 to 65 weight % of a humectant, e.g., 10 to 60 weight % of a humectant, e.g., 10 to 50 weight % of a humectant, e.g., 20 to 50 weight % of at a humectant, e.g., 20 to 40 weight % of a humectant. Humectants that may be used in the oral care compositions disclosed herein, e.g., any of Composition 1, et seq., include, for example, glycerin, sorbitol, xylitol, butylene glycol, polyethylene glycol, propylene glycol, trimethyl glycine, and mixtures thereof. In some embodiments, a mixture of glycerin, sorbitol, and propylene glycol is used as the humectant in the oral care compositions disclosed herein, e.g., any of Composition 1, et seq. In some embodiments, the oral care compositions disclosed herein, e.g., any of Composition 1, et seq., comprise, on a pure humectant basis, from 0 to 70 weight % of glycerin, sorbitol, and propylene glycol, e.g., 10 to 70 weight % of glycerin, sorbitol, and propylene glycol, e.g., 10 to 65 weight % of glycerin, sorbitol, and propylene glycol, e.g., 10 to 60 weight % of glycerin, sorbitol, and propylene glycol, e.g., 10 to 50 weight % of glycerin, sorbitol, and propylene glycol, e.g., 20 to 50 weight % of glycerin, sorbitol, and propylene glycol, e.g., 20 to 40 weight % of glycerin, sorbitol, and propylene glycol.

In some embodiments, the oral care compositions disclosed herein, e.g., any of Composition 1, et seq., comprise a surfactant, e.g., selected from anionic, cationic, zwitterionic, and nonionic surfactants, and mixtures thereof. In some embodiments, the surfactant is reasonably stable throughout a wide pH range. In some embodiments, the oral care compositions disclosed herein, e.g., any of Composition 1, et seq., comprise from 0.01 to 10 weight % of a surfactant, e.g., 0.05 to 5 weight % of a surfactant, e.g., 0.1 to 10 weight % of a surfactant, e.g., 0.1 to 5 weight % of a surfactant, e.g., 0.1 to 2 weight % of a surfactant, e.g., 0.5 to 2 weight % of a surfactant. In some embodiments, the oral care compositions disclosed herein, e.g., any of Composition 1, et seq., comprise from 0.01 to 10 weight % of an anionic surfactant, e.g., 0.05 to 5 weight % of an anionic surfactant, e.g., 0.1 to 10 weight % of an anionic surfactant, e.g., 0.1 to 5 weight % of an anionic surfactant, e.g., 0.1 to 2 weight % of an anionic surfactant, e.g., 0.5 to 2 weight % of an anionic surfactant, e.g., 1.5 weight % of an anionic surfactant.

Anionic surfactants that may be used in the oral care compositions disclosed herein, e.g., any of Composition 1, et seq., include, for example,
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl sulfates, such as sodium lauryl sulfate,
iii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate, CH₃(CH₂)₁₀CH₂(OCH₂CH₂)₂OSO₃Na,
iv. higher alkyl aryl sulfonates such as sodium dodecyl benzene sulfonate (sodium lauryl benzene sulfonate), and
v. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

As used herein, “higher alkyl” refers to C6-30 alkyl.

In some embodiments, the oral care compositions disclosed herein, e.g., Composition 1, et seq., comprise an anionic surfactant. In some embodiments, the anionic surfactant is the water soluble salt of alkyl sulfates having from 10 to 18 carbon atoms in the alkyl radical and water soluble salts of sulfonated monoglycerides of fatty acids having from 10 to 18 carbon atoms. Sodium lauryl sulfate, sodium lauroyl sarcosinate, and sodium coconut monoglyceride sulfonates are examples of anionic surfactants of that type. In some embodiments, the oral care compositions disclosed herein, e.g., any of Composition 1, et seq., comprise sodium lauryl sulfate, sodium ether lauryl sulfate, or a mixture thereof. In some embodiments, the oral care compositions disclosed herein, e.g., any of Composition 1, et seq., comprise sodium lauryl sulfate. In some embodiments, the oral care compositions disclosed herein, e.g., any of Composition 1, et seq., comprise from 0.01 to 10 weight % sodium lauryl sulfate, e.g., 0.05 to 5 weight % sodium lauryl sulfate, e.g., 0.1 to 10 weight % sodium lauryl sulfate, e.g., 0.1 to 5 weight %, e.g., sodium lauryl sulfate, e.g., 0.1 to 2 weight % sodium lauryl sulfate, e.g., 0.5 to 2 weight % sodium lauryl sulfate, e.g., 1.5 weight % sodium lauryl sulfate.

An abrasive removes debris and surface stains. In some embodiments, the oral care compositions disclosed herein, e.g., any of Composition 1, et seq., comprise 5 to 70 weight % of an abrasive, e.g., 5 to 60 weight % of an abrasive, e.g., 5 to 50 weight % of an abrasive, e.g., 5 to 40 weight % of an abrasive, e.g., 5 to 30 weight % of an abrasive, e.g., 10 to 30 weight % of an abrasive, e.g., 10 to 20 weight % of an abrasive.

Abrasives that may be used in the oral care compositions disclosed herein, e.g., any of Composition 1, et seq., include, for example, a calcium phosphate abrasive, e.g., tricalcium phosphate (Ca₃(PO₄)₂), hydroxyapatite (Ca₁₀(PO₄)₆(OH)₂), dicalcium phosphate dihydrate (CaHPO₄-2H₂O, also sometimes referred to herein as DiCal), calcium pyrophosphate, and mixtures thereof. Calcium carbonate, e.g., precipitated calcium carbonate, may also be employed as an abrasive.

Other abrasives that may be used in the oral care compositions disclosed herein, e.g., any of Composition 1, et seq., include, for example, silica abrasives such as precipitated silicas having a mean particle size of up to about 20 microns, such as Zeodent 115®, marketed by J. M. Huber, as well as sodium metaphosphate, potassium metaphosphate, aluminum silicate, calcined alumina, bentonite or other siliceous materials, or mixtures thereof. Silica abrasives used herein, as well as the other abrasives, may have an average particle size ranging between about 0.1 and about 30 microns, e.g., between about 5 and about 15 microns. The silica abrasives may be from precipitated silica or silica gels, such as silica xerogels. Particular silica xerogels are marketed under the trade name Syloid® by the W. R. Grace & Co. Davison Chemical Division. Precipitated silica materials include those marketed by the J. M. Huber Corp. under the trade name Zeodent®, including the silica carrying the designation Zeodent 115 and 119.

In some embodiments, abrasives that may be used in the oral care compositions disclosed herein, e.g., any of Composition 1, et seq., include silica gels and precipitated amorphous silica having an oil absorption value of about less than about 100 cc/100 g silica and in the range of about 45 cc/100 g to about 70 cc/100 g silica. Oil absorption values are measured using the ASTA Rub-Out Method D281. In some embodiments, the silica comprises colloidal particles having an average particle size of about 3 microns to about 12 microns, and about 5 to about 10 microns.

In some embodiments, the abrasive comprises a large fraction of very small particles, e.g., having a d50 less than about 5 microns, e.g., small particle silica (SPS) having a d50 of about 3 to about 4 microns, e.g., Sorbosil AC AC43® (Ineos). Such small particles may be used in formulations targeted at reducing hypersensitivity. The small particle component may be present in combination with a second larger particle abrasive.

Low oil absorption silica abrasives that may be used in the oral care compositions disclosed herein, e.g., any of Composition 1, et seq., are marketed under the trade designation Sylodent WXA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive that may be used in the oral care compositions disclosed herein, e.g., any of Composition 1, et seq.

In some embodiments, the oral care composition disclosed herein, e.g., any of Composition 1, comprise a high cleaning silica. In some embodiments, the oral care compositions disclosed herein, e.g., any of Composition 1, et seq., comprise 5 to 70 weight % high cleaning silica, e.g., 5 to 60 weight % high cleaning silica, e.g., 5 to 50 weight % high cleaning silica, e.g., 5 to 40 weight % high cleaning silica, e.g., 5 to 30 weight % high cleaning silica, e.g., 10 to 30 weight % high cleaning silica, e.g., 10 to 20 weight % high cleaning silica.

In some embodiments, the oral care compositions disclosed herein, e.g., any of Composition 1, et seq., comprise a sweetener. In some embodiments, the oral care compositions disclosed herein, e.g., any of Composition 1, et seq., comprise 0.005 to 10 weight % of a sweetener, e.g., 0.01 to 10 weight % of a sweetener, e.g., 0.1 to 10 weight % of a sweetener, e.g., from 0.1 to 5 weight % of a sweetener, e.g., from 0.1 to 3 weight % of a sweetener, e.g., from 0.1 to 1 weight % of a sweetener, e.g., from 0.1 to 0.5 weight % of a sweetener. Sweeteners that may be used in the oral care compositions disclosed herein, e.g., any of Composition 1, et seq., include, for example, sucrose, glucose, saccharin, sucralose, dextrose, levulose, lactose, mannitol, sorbitol, fructose, maltose, xylitol, saccharin salts (e.g., sodium saccharin), thaumatin, aspartame, D-tryptophan, dihydrochalcones, acesulfame, cyclamate salts, and mixtures thereof. In some embodiments, sodium saccharin is used as the sweetener in the oral care compositions disclosed herein, e.g., any of Composition 1, et seq. In some embodiments, the oral care compositions disclosed herein, e.g., any of Composition 1, et seq., comprise 0.005 to 10 weight % sodium saccharin, e.g., 0.01 to 10 weight % sodium saccharin, e.g., 0.1 to 10 weight % sodium saccharin, e.g., from 0.1 to 5 weight % sodium saccharin, e.g., from 0.1 to 3 weight % sodium saccharin, e.g., from 0.1 to 1 weight % sodium saccharin, e.g., from 0.1 to 0.5 weight % sodium saccharin.

In some embodiments, the oral care compositions disclosed herein, e.g., any of Composition 1, et seq., comprise a flavorant. In some embodiments, the oral care compositions disclosed herein, e.g., any of Composition 1, et seq., comprise 0.1 to 5 weight % of a flavorant, e.g., 0.1 to 4 weight % of a flavorant, e.g., 0.1 to 3 weight % of a flavorant, e.g., 0.1 to 2 weight % of a flavorant, e.g., 0.5 to 2 weight % of a flavorant, e.g., 0.6 to 2 weight % of a flavorant, e.g., 0.7 to 2 weight % of a flavorant, e.g., 0.8 to 2 weight % of a flavorant e.g., 0.9 to 2 weight % of a flavorant, e.g., 1 to 2 weight % of a flavorant. Flavorants that may be used in the oral care compositions disclosed herein, e.g., any of Composition 1, et seq., include, for example, essential oils, as well as various flavoring aldehydes, esters, alcohols, and similar materials, as well as menthol, carvone, and anethole, as well as mixtures thereof. Examples of essential oils include oils of spearmint, peppermint, wintergreen, sassafras, clove, sage, eucalyptus, marjoram, cinnamon, lemon, lime, grapefruit, and orange. In some embodiments, a mixture of peppermint oil and spearmint oil is used as the flavorant in the oral care compositions disclosed herein, e.g., Composition 1, et seq.

In some embodiments, the oral care compositions disclosed herein, e.g., any of Composition 1, et seq., comprise a pigment. In some embodiments, the oral care compositions disclosed herein, e.g., any of Composition 1, et seq., comprise 0.001 to 20 weight % of a pigment, e.g., 0.01 to 20 weight % of a pigment, e.g., 0.01 to 20 weight % of a pigment, e.g., 0.1 to 20 weight % of a pigment, e.g., 0.1 to 10 weight % of a pigment, e.g., 0.1 to 5 weight % of a pigment, e.g., 0.1 to 3 weight % of a pigment, e.g., 0.1 to 1 weight % of a pigment. In some embodiments, the oral care compositions disclosed herein, e.g., any of Composition 1, et seq., comprise titanium dioxide. In some embodiments, the oral care compositions disclosed herein, e.g., any of Composition 1, et seq., comprise 0.001 to 20 weight % titanium dioxide, e.g., 0.01 to 20 weight % titanium dioxide, e.g., 0.01 to 20 weight % titanium dioxide, e.g., 0.1 to 20 weight % titanium dioxide, e.g., 0.1 to 10 weight % titanium dioxide, e.g., 0.1 to 5 weight % titanium dioxide, e.g., 0.1 to 3 weight % titanium dioxide, e.g., 0.1 to 1 weight % titanium dioxide.

In some embodiments, the oral care compositions disclosed herein, e.g., any of Composition 1, et seq., further comprise an anti-caries agent. In some embodiments, the oral care compositions disclosed herein, e.g., any of Composition 1, et seq., comprise 0.005 to 10 weight % of the anti-caries agent, e.g., 0.01 to 10 weight % of the anti-caries agent, e.g., 0.01 to 5 weight % of the anti-caries agent, e.g., 0.01 to 1 weight % of the anti-caries agent, e.g., 0.01 to 0.3 weight % of the anti-caries agent, e.g., 0.1 to 10 weight % of the anti-caries agent, e.g., 0.1 to 5 weight % of the anti-caries agent, e.g., 0.1 to 2 weight % of the anti-caries agent, e.g., 0.1 to 1 weight % of the anti-caries agent, e.g., 0.1 to 0.8 weight % of the anti-caries agent, e.g., 0.1 to 0.6 weight % of the anti-caries agent, e.g., 0.1 to 0.5 weight % of the anti-caries agent. In some embodiments, the anti-caries agent is a fluoride ion source. In some embodiments, the oral care compositions disclosed herein, e.g., any of Composition 1, et seq., further comprise 0.005 to 10 weight % of the anti-caries agent which is a fluoride ion source, e.g., 0.01 to 10 weight % of the anti-caries agent which is a fluoride ion source, e.g., 0.01 to 5 weight % of the anti-caries agent which is a fluoride ion source, e.g., 0.01 to 1 weight % of the anti-caries agent which is a fluoride ion source, e.g., 0.01 to 0.3 weight % of the anti-caries agent which is a fluoride ion source, e.g., 0.1 to 10 weight % of the anti-caries agent which is a fluoride ion source, e.g., 0.1 to 5 weight % of the anti-caries agent which is a fluoride ion source, e.g., 0.1 to 2 weight % of the anti-caries agent which is a fluoride ion source, e.g., 0.1 to 1 weight % of the anti-caries agent which is a fluoride ion source, e.g., 0.1 to 0.8 weight % of the anti-caries agent which is a fluoride ion source, e.g., 0.1 to 0.6 weight % of the anti-caries agent which is a fluoride ion source, e.g., 0.1 to 0.5 weight % of the anti-caries agent which is a fluoride ion source. Examples of fluoride ion sources that may be used in the oral compositions disclosed herein, e.g., any of Composition 1, et seq., are found in U.S. Pat. No. 3,535,421 to Briner et al.; U.S. Pat. No. 4,885,155 to Parran, Jr. et al., and U.S. Pat. No. 3,678,154 to Widder et al, incorporated herein by reference in their entirety. Other examples of fluoride ion sources include, for example, stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In certain embodiments the fluoride ion source includes stannous fluoride, sodium fluoride, and sodium monofluorophosphate, as well as mixtures thereof. In some embodiments, the anti-caries agent is sodium fluoride. In some embodiments, the oral care compositions disclosed herein, e.g., any of Composition 1, et seq., comprise 0.005 to 10 weight % sodium fluoride, e.g., 0.01 to 10 weight % sodium fluoride, e.g., 0.01 to 5 weight % sodium fluoride, e.g., 0.01 to 1 weight % sodium fluoride, e.g., 0.01 to 0.3 weight % sodium fluoride, e.g., 0.1 to 10 weight % sodium fluoride, e.g., 0.1 to 5 weight % sodium fluoride, e.g., 0.1 to 2 weight % sodium fluoride, e.g., 0.1 to 1 weight % sodium fluoride, e.g., 0.1 to 0.8 weight % sodium fluoride, e.g., 0.1 to 0.6 weight % sodium fluoride, e.g., 0.1 to 0.5 weight % sodium fluoride.

In some embodiments, the oral care compositions disclosed herein, e.g., any of Composition 1, et seq., comprise the anti-caries agent which is a fluoride ion source in an amount sufficient to supply 25 ppm to 25,000 ppm of fluoride ions, e.g., from 100 to 20,000 ppm of fluoride ions, e.g., from 300 to 15,000 ppm of fluoride ions, e.g., from 500 to 10,000 ppm of fluoride ions, e.g., from 500 to 8,000 ppm of fluoride ions, e.g., from 500 to 6,000 ppm of fluoride ions, e.g., from 500 to 4,000 ppm of fluoride ions, e.g., from 500 to 2,000 ppm of fluoride ions, e.g., from 500 to 1,800 ppm of fluoride ions, e.g., from 1000 to 1600 ppm, e.g., 1450 ppm of fluoride ions. The appropriate level of fluoride ions will depend on the particular application. In some embodiments, a toothpaste for consumer use comprises the anti-caries agent which is a fluoride ion source in an amount sufficient to supply from 1,000 to 1,500 ppm of fluoride ions, with pediatric toothpaste having somewhat less. In some embodiments, a dentifrice or coating for professional application comprises the anti-caries agent which is a fluoride ion source in an amount sufficient to supply from 5,000 to 25,000 ppm of fluoride ions.

A whitening agent whitens a tooth to which it is applied. In some embodiments, the oral care compositions disclosed herein, e.g., any of Composition 1, et seq., comprise a whitening agent. In some embodiments, the oral care compositions disclosed herein, e.g., any of Composition 1, et seq., comprise a whitening agent in a dental surface-whitening effective amount, e.g., 0.1 to 90 weight % whitening agent, e.g., 0.5 to 50 weight % whitening agent, e.g., 1 to 30 weight % whitening agent, e.g., 2 to 10 weight % whitening agent. Examples of whitening agents that may be used in the oral compositions disclosed herein, e.g., any of Composition 1, et seq., include, for example, peroxides, metal chlorites, perborates, percarbonates, peroxyacids, hypochlorites, and mixtures thereof. In some embodiments, the whitening agent is hydrogen peroxide or a hydrogen peroxide source, for example, urea peroxide or a peroxide salt or complex (for example, peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or a hydrogen peroxide polymer complex (for example, a peroxide-polyvinyl pyrrolidone polymer complex).

The following examples are further illustrative of the nature of the present invention, but it is understood that the invention is not limited thereto. All amounts and proportions referred to herein and in the appended claims are by weight, unless otherwise indicated.

### EXAMPLES

### Example 1 - Effects of Substrates on Multi-Species Biofilm Composition

Biofilms are grown vertically on Calcium Deficient Hydroxyapatite (CAD-HA) disks (Hitemco Medical, Old Bethpage, USA) using the Amsterdam Active Adhesion model. Biofilms are allowed to establish during 24 h (37° C., 170 rpm) under microaerophilic conditions (6% O₂, 7% CO₂, 7% H₂, 80% N₂). After this 24 h, disks are rinsed 3 times a day for 3 minutes (RT, 250 rpm), for 3 consecutive days, by transferring the lid containing the disks to a new 24-well plate containing 2 mL/well of the appropriate substrate solution. As a negative control, PBS (pH 5.7) without substrate supplementation is used.

N-acetyl-D-glucosamine (NADG), α-D-lactose, D-(+)-trehalose and D-(+)-raffinose are selected to determine their effects on multi-species biofilm composition. Repeated rinsing of the biofilms with the substrates at a concentration of 1 M results in a significant decrease in absolute numbers in at least four of the pathogenic bacterial species tested in the assay, in comparison to the control, the results of which are described in Table 1 below. Note, NADG decreased absolute numbers of S. sobrinus. Absolute abundances of bacterial species are shown as mean ± SD (n = 3) logarithmic values of the genome equivalents per millilitre (log(Geq/mL)). All substrates are dissolved in PBS at a concentration of 1 M. Statistically significantly different values in comparison to the control (PBS) are marked with an asterisk ‘*’ (P < 0.05):

TABLE 1

mean ± SD log (Geq/mL)

| 1 M | control | NADG | D-lactose | D-trehalose | D-raffinose |
|---|---|---|---|---|---|
| Aa | 9.27 ± 0.31 | 8.15 ± 0.21^{∗} | 7.87 ± 0.04^{∗} | 7.86 ± 0.22^{∗} | 8.30 ± 0.29^{∗} |
| Fn | 9.48 ± 0.12 | 7.22 ± 0.28^{∗} | 5.49 ± 0.46^{∗} | 6.51 ± 0.43^{∗} | 6.73 ± 0.06^{∗} |
| Pg | 6.97 ± 0.04 | 4.43 ± 0.14^{∗} | 4.26 ± 0.51^{∗} | 4.37 ± 0.29^{∗} | 4.59 ± 0.45^{∗} |
| Pi | 7.23 ± 0.47 | 4.09 ± 0.16^{∗} | 4.05 ± 0.61^{∗} | 4.47 ± 0.60^{∗} | 5.21 ± 0.77^{∗} |
| S. mutans | 7.16 ± 0.42 | 7.96 ± 0.10 | 8.00 ± 0.56 | 8.50 ± 0.29^{∗} | 8.24 ± 0.17^{∗} |
| S. sobrinus | 6.50 ± 0.43 | 3.46 ± 0.19^{∗} | 7.82 ± 0.11^{∗} | 7.44 ± 0.18^{∗} | 7.06 ± 0.15 |
| 1 M | control | NADG | D-lactose | D-trehalose | D-raffinose |
| An | 7.28 ± 0.18 | 5.41 ± 0.31^{∗} | 4.86 ± 0.19^{∗} | 4.81 ± 0.47^{∗} | 5.32 ± 0.25^{∗} |
| Av | 8.29 ± 0.82 | 6.72 ± 0.22 | 6.78 ± 0.41 | 6.05 ± 1.12^{∗} | 7.08 ± 0.59 |
| S. gord. | 8.75 ± 0.22 | 9.80 ± 0.22^{∗} | 9.31 ± 0.41 | 9.39 ± 0.31 | 8.93 ± 0.31 |
| S. mitis | 2.90 ± 0.36 | 3.38 ± 1.04 | 3.17 ± 0.75 | 3.15 ± 0.71 | 3.58 ± 1.31 |
| S. oralis | 7.25 ± 0.49 | 7.96 ± 0.59 | 7.45 ± 0.46 | 6.71 ± 0.48 | 8.52 ± 0.17^{∗} |
| S. sal. | 5.46 ± 0.33 | 6.40 ± 0.29^{∗} | 6.95 ± 0.14^{∗} | 6.19 ± 0.07^{∗} | 6.29 ± 0.21^{∗} |
| S. sang. | 6.65 ± 0.64 | 6.43 ± 0.94 | 5.80 ± 1.09 | 6.43 ± 0.97 | 6.39 ± 0.51 |
| V_{P} | 10.14 ± 0.04 | 9.91 ± 0.19 | 9.45 ± 0.15^{∗} | 9.44 ± 0.25^{∗} | 9.88 ± 0.07 |

Note: “Aa”: A. actinomycetemcomitans; “Fn”: F. nucleatum; “Pg”: P. gingivalis; “Pi”: P. intermedia; “An”: A. naeslundii; “Av:” A. viscosus; S. gord.: S. gordonii; S. sal.: S. salivarius; S. sang.: S. sanguinis; Vp: V. parvula; NADG: N-acetyl-D-glucosamine.

These decreases (expressed as logarithmic value of the amount of genome equivalents per millilitre; log(Geq/mL)) are approximately:
~1 to 1.4 log(Geq/mL) for A. actinomycetemcomitans,
~2.3 to 4 log(Geq/mL) for F. nucleatum,
~2.4 to 2.7 log(Geq/mL) for P. gingivalis and;
~2 to 3.2 log(Geq/mL) for P. intermedia.

The numbers of the cariogenic pathogens S. mutans and S. sobrinus are usually increased with ~0.6-1.3 log(Geq/mL) in comparison to the control, with statistical significance being reached for S. mutans in the case of D-(+)-trehalose and D-(+)-raffinose, and for S. sobrinus in the case of α-D-lactose and D-(+)-trehalose. However, surprisingly, NADG results in a significant decrease in S. sobrinus numbers with ~3 log(Geq/mL). Table 1 indicates that the numbers of the beneficial/commensal species A. naeslundii (for all substrates) and A. viscosus (for D-(+)-trehalose) are often significantly decreased with ~1.9-2.5 log(Geq/mL), and V. parvula showed significant reductions of ~0.7 log(Geq/mL) for α-D-lactose and D-(+)-trehalose.

Table 1 also demonstrates, with respect to the often beneficial streptococcal species, the bacterial numbers generally increased with ~0.2 to 1.3 log(Geq/mL), except for S. sanguinis, with statistical significance being reached for S. gordonii (using NADG), S. oralis (using D-(+)-raffinose) and S. salivarius (using any of the four prebiotic substrates).

In terms of relative proportions (%(Geq/mL)), the control treatment results in a biofilm consisting of 74.21±6.47% beneficial/commensal species, 25.67±6.52% periodontal pathogens and 0.13±0.06% cariogenic pathogens. Rinsing with the substrates results in significantly lower proportions of periodontal pathogens (1.46±0.97%, 1.46±0.64%, 1.80±1.04% and 2.29±1.40% for NADG, α-D-lactose, D-(+)-trehalose and D-(+)-raffinose, respectively) in comparison to the control condition. All substrates also results in significantly higher proportions of beneficial/commensal species (97.86±1.11%, 92.72±4.39%, 88.98±9.12% and 95.86±1.63% for NADG, α-D-lactose, D-(+)-trehalose and D-(+)-raffinose, respectively). Proportions of cariogenic pathogens are elevated in all cases (0.68±0.33%, 5.82±3.94%, 9.22±8.55% and 1.85±0.68% for NADG, α-D-lactose, D-(+)-trehalose and D-(+)-raffinose, respectively), although not significantly in comparison to the control. This is detailed in Table 1a (Statistically significantly different values in comparison to the control are marked with an asterisk ‘*’ (P < 0.05)):

TABLE 1a

| % (Geq/mL) | Beneficial or commensals | Periodontal Pathogens | Cariogenic Pathogens |
|---|---|---|---|
| Control | 74.21 (+/- 6.47) | 25.67 (+/- 6.52) | 0.13 (+/- 0.06) |
| NADG | 97.86 (+/- 1.11)^{∗} | 1.46 (+/- 0.97)^{∗} | 0.68 (+/- 0.33) |
| D-lactose | 92.72 (+/- 4.39)^{∗} | 1.46 (+/- 0.64)^{∗} | 5.82 (+/- 3.94) |
| D-trehalose | 88.98 (+/- 9.12)^{∗} | 1.80 (+/- 1.04)^{∗} | 9.22 (+/- 8.55) |
| D-raffinose | 95.86 (+/- 1.63)^{∗} | 2.29 (+/- 1.40)^{∗} | 1.85 (+/- 0.68) |

When rinsing the biofilms with the substrates at a concentration of 1% (_{w/v}), absolute numbers of beneficial/commensal species, periodontal pathogens and cariogenic pathogens are, generally, slightly affected in comparison to the control (changes of ~0-0.6 log(Geq/mL), though this did not reach statistical significance. However, in the case of S. oralis, there is a significant decrease of ~0.9 log(Geq/mL) for D-(+)-raffinose. There is a trend toward higher proportions of beneficial or commensal species and lower proportions of periodontal pathogens. The results are detailed in Table 2:

TABLE 2

mean ± SD log (Geq/mL)

| 1% (w/v) | control | NADG | D-lactose | D-trehalose | D-raffinose |
|---|---|---|---|---|---|
| Aa | 9.49 ± 0.25 | 9.11 ± 0.2 | 9.44 ± 0.41 | 9.28 ± 0.43 | 9.14 ± 0.12 |
| Fn | 9.71 ± 0.11 | 9.41 ± 0.19 | 9.39 ± 0.42 | 9.35 ± 0.20 | 9.40 ± 0.10 |
| Pg | 7.27 ± 0.20 | 7.14 ± 0.28 | 6.93 ± 0.10 | 7.05 ± 0.19 | 6.94 ± 0.21 |
| Pi | 8.02 ± 0.24 | 8.17 ± 0.22 | 8.13 ± 0.25 | 7.88 ± 0.33 | 7.83 ± 0.32 |
| S. mutans | 7.48 ± 0.13 | 7.83 ± 0.23 | 7.48 ± 0.14 | 7.55 ± 0.25 | 7.46 ± 0.16 |
| S. sobrinus | 5.16 ± 0.07 | 4.87 ± 0.22 | 5.27 ± 0.24 | 5.27 ± 0.19 | 5.36 ± 0.20 |
| 1% (w/v) | control | NADG | D-lactose | D-trehalose | D-raffinose |
| An | 7.58 ± 0.13 | 7.71 ± 0.12 | 7.75 ± 0.24 | 7.6 ± 0.15 | 7.62 ± 0.08 |
| Av | 8.01 ± 0.44 | 7.83 ± 0.31 | 7.89 ± 0.13 | 8.04 ± 0.28 | 7.77 ± 0.14 |
| S. gord. | 8.33 ± 0.18 | 8.33 ± 0.06 | 8.4 ± 0.11 | 8.45 ± 0.27 | 8.12 ± 0.06 |
| S. mitis | 2.65 ± 0.00 | 2.65 ± 0.00 | 2.65 ± 0.00 | 2.65 ± 0.00 | 2.65 ± 0.00 |
| S. oralis | 6.9 ± 0.2 | 7.11 ± 0.14 | 6.6 ± 0.17 | 6.59 ± 0.42 | 5.98 ± 0.18^{∗} |
| S. sal. | 2.65 ± 0.00 | 2.65 ± 0.00 | 2.65 ± 0.00 | 2.65 ± 0.00 | 2.65 ± 0.00 |
| S. sang. | 8.37 ± 0.25 | 8.40 ± 0.11 | 8.99 ± 0.54 | 8.42 ± 0.43 | 8.56 ± 0.11 |
| V_{P} | 10.07 ± 0.16 | 9.77 ± 0.19 | 9.92 ± 0.20 | 10.07 ± 0.28 | 9.83 ± 0.09 |

Note: “Aa”: A. actinomycetemcomitans; “Fn”: F. nucleatum; “Pg”: P. gingivalis; “Pi”: P. intermedia; “An”: A. naeslundii; “Av:” A. viscosus; S. gord.: S. gordonii; S. sal.: S. salivarius; S. sang.: S. sanguinis; Vp: V. parvula; NADG: N-acetyl-D-glucosamine.

As detailed in Table 2, when rinsing the biofilms with the substrates at a concentration of 1% _{(w/v)}, absolute numbers of beneficial or commensal species, periodontal pathogens and cariogenic pathogens are in general only slightly affected in comparison to the control (changes of ~0-0.6 log (Geq/mL), without reaching statistical significance. Only in the case of S. oralis is there a significant decrease of ~0.9 log (Geq/mL) for D-(+)-raffinose. There are tendencies for higher proportions of beneficial/commensal species and lower proportions of periodontal pathogens but no significant differences in comparison to the control condition.

Separate experiments provide confirmatory results for the proportions of: beneficial/commensal bacteria, periodontal pathogens, and cariogenic pathogens, in biofilm upon treatment with a control or a particular prebiotic saccharide. As shown in Table 3, the proportions of cariogenic pathogens are usually unchanged, except for those in the NADG condition, which results in a significantly higher proportion of cariogenic species (0.66±0.27%) in comparison to the control (0.16±0.06%). This is detailed in Table 3:

TABLE 3

| % (Geq/mL) | Beneficials/Commensals | Periodontal Pathogens | Cariogenic Pathogens |
|---|---|---|---|
| Control | 59.22 (+/- 1.17) | 40.63 (+/- 1.13) | 0.16 (+/- 0.06) |
| NADG | 60.07 (+/- 8.25) | 39.27 (+/- 7.98) | 0.66 (+/- 0.27^{∗}) |
| D-lactose | 64.14 (+/- 9.81) | 35.65 (+/- 9.88) | 0.21 (+/- 0.08) |
| D-trehalose | 73.55 (+/- 2.84) | 26.23 (+/- 2.88) | 0.21 (+/- 0.05) |
| D-raffinose | 64.27 (+/- 1.84) | 35.46 (+/- 1.82) | 0.27 (+/- 0.09) |

### Example 2 - Effects of Substrates on Multi-Species Biofilm Organic Acid Balances

Levels of organic acids in the filter sterilized supernatant of the multi-species biofilms are measured using a 761 Compact Ion Chromatograph (Metrhohm, Switzerland) with a Metrosep Organic acids Guard/4.6 guard column. The supernatants from the substrate-treated multi-species biofilms are analyzed to gain more insight into the influence of the substrates on organic acid consumption/production. Table 4 below demonstrates that for substrate concentrations of 1 M, there is a significantly increased lactate production in the case of α-D-lactose (3791±169 mg/L), D-(+)-trehalose (4187±200 mg/L) and D-(+)-raffinose (971±43 mg/L) in comparison to the control condition (consumption of 125±0 mg/L). Formate production is significantly lower for α-D-lactose and D-(+)-trehalose in comparison with the control condition (84±9 mg/L and 75±8 mg/L vs. 384±19 mg/L). α-D-lactose (881±73 mg/L), D-(+)-trehalose (1021±73 mg/L) and D-(+)-raffinose (2640±154 mg/L) all results in significantly lower acetate production in comparison to the control condition (3779±305 mg/L). NADG, α-D-lactose, D-(+)-trehalose and D-(+)-raffinose all results in a significantly different production of propionate (2750±40 mg/L, 1306±175 mg/L, 1577±46 mg/L and 4178±105 mg/L, respectively) and butyrate (1255±51 mg/L, 0±0 mg/L, 0±0 mg/L and 154±26 mg/L, respectively) in comparison with the control condition (2094±132 mg/L propionate and 1870±93 mg/L butyrate).

Rinsing with substrates at concentrations of 1% _{(w/v)} does not result in significant differences in organic acid production/consumption in comparison to the control as demonstrated in Table 4:

TABLE 4

Mean ± SD organic acid levels (mg/L)

| 1 M | control | NADG | D-lactose | D-trehalose | D-raffinose |
|---|---|---|---|---|---|
| lactate | -125 ± 0 | -125 ± 0 | 3791 ± 169^{∗} | 4187 ± 200^{∗} | 971 ± 43^{∗} |
| formate | 384 ± 19 | 352 ± 18 | 84 ± 9^{∗} | 75 ± 8^{∗} | 344 ± 25^{∗} |
| acetate | 3779 ± 305 | 3815 ± 77 | 881 ± 73^{∗} | 1021 ± 41^{∗} | 2640 ± 154^{∗} |
| propionate | 2094 ± 132 | 2750 ± 40^{∗} | 1306 ± 175^{∗} | 1577 ± 46^{∗} | 4178 ± 105^{∗} |
| butyrate | 1870 ± 93 | 1255 ± 51^{∗} | 0±0^{∗} | 0±0^{∗} | 154 ± 26^{∗} |
| 1% (w/v) | control | NADG | D-lactose | D-trehalose | D-raffinose |
| lactate | -122 ± 5 | -125 ± 0 | -125 ± 0 | -125 ± 0 | -125 ± 0 |
| acetate | 345 ± 63 | 379 ± 10 | 367 ± 33 | 303 ± 13 | 380 ± 23 |
| formate | 3954 ± 539 | 4315 ± 198 | 3929 ± 253 | 3691 ± 89 | 3748 ± 197 |
| propionate | 2077 ± 234 | 2205 ± 89 | 2140 ± 141 | 1962 ± 8 | 2033 ± 48 |
| butyrate | 2129 ± 240 | 2238 ± 59 | 2061 ± 121 | 1945 ± 9 | 2003 ± 81 |

Organic acid levels detected in the supernatants of substrate-treated multi-species biofilms are shown as mean ± SD (n = 3) values (mg/L). Values >0 mg/L represent organic acid production, values <0 mg/L represent organic acid consumption.
Substrates are dissolved in PBS at a concentration of 1 M (upper part) or 1% (w/v) (lower part). Statistically significantly different values in comparison to the control (PBS) are marked with an asterisk ‘^{∗}’ (P < 0.05)

### Example 3 - Effects of Substrates on Multi-Species Biofilm Virulence Gene Expression

The virulence of the substrate-treated multi-species biofilms are evaluated by analyzing the relative expression of 33 genes that are recognized as being related to some form of virulence and from 4 periodontal pathogens.

Significantly different gene expressions in the substrate-treated biofilms relatively to the control biofilms are considered to be biologically relevant if their value is more than 1.5-fold upregulated or more than 2-fold downregulated. Only these results are considered.

Expression of bacterial virulence genes is analyzed through SYBR RT-qPCR and normalized for bacterial housekeeping gene expression. In multi-species biofilms rinsed with the substrates at concentrations of 1 M, A. actinomycetemcomitans and P. gingivalis virulence gene expressions are significantly downregulated relatively to the control condition for most substrates. These effects are illustrated in Tables 6a and 6b:

Table 6a - Effects of repeated rinsing of multi-species biofilms with potentially prebiotic substrates on virulence gene expression from A. actinomycetemcomitans (Statistically significantly different values in comparison to the control are marked with an asterisk ‘*’ (P < 0.05)):

Fold Change in virulence gene expression as measured by SYBR RT-qPCR^{∗∗}

| Genes | NADG 1 M | D-lactose 1 M | D-trehalose 1 M | D-raffinose 1 M |
|---|---|---|---|---|
| flp | 2.40 (0.67-8.67) | 0.44 (0.10-1.94) | 0.71 (0.30-1.67) | 0.45 (0.17-1.22) |
| aae | 0.24 (0.18-0.33)^{∗} | 0.03 (0.01-0.22)^{∗} | 0.03 (0.01-0.15)^{∗} | 0.16 (0.05-0.47)^{∗} |
| apaH | 0.08 (0.02-0.29)^{∗} | 0.04 (0.01-0.21)^{∗} | 0.16 (0.04-0.56)^{∗} | 0.11 (0.02-0.73)^{∗} |
| cdtB | 0.04 (0.01-0.20)* | 0.02 (0.00-0.21)^{∗} | 0.08 (0.03-0.21)^{∗} | 0.07 (0.02-0.25)^{∗} |
| emaA | 0.29 (0.07-1.20)* | 0.08 (0.06-0.11)^{∗} | 0.11 (0.04-0.32)^{∗} | 0.09 (0.03-0.29)^{∗} |
| ltxA | 0.01 (0.00-0.20)* | 0.01 (0.00-0.08)^{∗} | 0.01 (0.00-0.01)^{∗} | 0.01 (0.01-0.03)^{∗} |
| omp100 | 0.03 (0.01-0.05)* | 0.08 (0.04-0.15)^{∗} | 0.09 (0.01-1.11)^{∗} | 0.09 (0.02-0.43)^{∗} |
| omp29 | 0.19 (0.09-0.38)* | 0.42 (0.33-0.52)^{∗} | 0.74 (0.18-3.00) | 1.37 (0.34-5.47) |
| orf859 | 0.13 (0.05-0.36)* | 0.05 (0.00-0.79)^{∗} | 0.14 (0.02-0.87)^{∗} | 0.02 (0.01-0.05)^{∗} |
| pgA | 11.6 (3.23-41.74)* | 4.30 (2.21-8.37)^{∗} | 18.24 (5.89-56.51)^{∗} | 7.26 (2.09-25.17)^{∗} |
| vapA | 1.00 (0.58-1.75) | 0.22 (0.13-0.36)^{∗} | 0.32 (0.08-1.26)^{∗} | 0.50 (0.13-1.94) |
| vppA | 0.49 (0.41-0.59) | 0.17 (0.06-0.43)^{∗} | 0.32 (0.01-4.50)^{∗} | 0.05 (0.04-2.42)^{∗} |
| flp | 0.54 (0.19-1.56) | 0.81 (0.08-7.83) | 0.48 (0.07-3.50) | 0.89 (0.40-1.99) |
| aae | 1.55 (1.18-2.03) | 1.36 (0.70-2.67) | 0.95 (0.33-2.78) | 1.34 (0.38-4.68) |
| apaH | 0.73 (0.41-1.29) | 0.84 (0.29-2.49) | 0.64 (0.37-1.10) | 0.91 (0.48-1.73) |
| cdtB | 1.05 (0.61-1.81) | 0.66 (0.21-2.03) | 0.67 (0.51-0.89) | 0.73 (0.16-3.39) |
| emaA | 0.68 (0.15-3.07) | 0.34 (0.25-0.45)^{∗} | 0.67 (0.49-0.90) | 0.59 (0.13-2.77) |
| ltxA | 0.4 (0.11-1.39) | 0.04 (0.02-0.08)^{∗} | 0.08 (0.01-0.73)^{∗} | 0.30 (0.12-0.79) |
| omp100 | 0.71 (0.42-1.18) | 0.34 (0.23-0.49)^{∗} | 0.43 (0.26-0.72)^{∗} | 0.31 (0.09-1.08)^{∗} |
| omp29 | 0.77 (0.69-0.87) | 0.81 (0.73-0.89) | 0.51 (0.19-1.34)^{∗} | 0.87 (0.26-2.87) |
| orf859 | 1.99 (0.94-4.22)* | 0.97 (0.37-2.54) | 1.04 (0.53-2.03) | 1.66 (0.86-3.20) |
| pgA | 1.07 (0.29-3.97) | 0.55 (0.41-0.74) | 0.91 (0.81-1.03) | 0.99 (0.38-2.55) |
| vapA | 0.65 (0.48-0.86) | 0.90 (0.60-1.35) | 1.02 (0.87-1.21) | 1.14 (0.40-3.20) |
| vppA | 0.28 (0.09-0.86)* | 0.40 (0.14-1.15)^{∗} | 0.48 (0.42-0.55)^{∗} | 1.08 (0.53-2.18) |

^{∗∗} Results are listed as fold changes relative to the control conditions (2^{^-ΔΔCt} method), the values for the control are normalized to “1”.

Table 6b - Effects of repeated rinsing of multi-species biofilms with potentially prebiotic substrates on virulence gene expression from P. gingivalis (Statistically significantly different values in comparison to the control are marked with an asterisk ‘*’ (P < 0.05)):

Fold Change in virulence gene expression as measured by SYBR RT-qPCR

| | NADG 1 M | D-lactose 1 M | D-trehalose 1 M | D-raffinose 1 M |
|---|---|---|---|---|
| kgp | 0.07 (0.04-0.13)^{∗} | <LOD | <LOD | <LOD |
| fimA | 0.79 (0.44-1.39) | 0.06 (0.00-1.08)^{∗} | 0.13 (0.02-0.96)^{∗} | 0.03 (0.00-0.16)* |
| partC | 0.19 (0.13-0.28)^{∗} | 0.03 (0.01-0.17)^{∗} | 0.03 (0.00-0.49)^{∗} | 0.02 (0.00-0.12)^{∗} |
| rgpA | 0.39 (0.18-0.84) | 0.01 (0.00-4.60)^{∗} | 0.00 (0.00-0.02)^{∗} | 0.01 (0.00-0.10)^{∗} |
| Genes | NADG 1 % (w/v) | D-lactose 1 % (w/v) | D-trehalose 1 % (w/v) | D-raffinose 1 % (w/v) |
| kgp | 1.81 (0.44-7.50) | 1.95 (0.45-8.59) | 3.24 (1.63-6.44)^{∗} | 1.77 (0.74-4.21) |
| fimA | 1.15 (0.32-4.06) | 1.48 (0.27-8.02) | 1.58 (0.86-2.90) | 1.47 (0.92-2.34) |
| partC | 0.36 (0.08-1.68)^{∗} | 0.47 (0.07-3.04) | 0.71 (0.37-1.35) | 0.54 (0.26-1.12) |
| rgpA | 1.31 (1.03-1.66) | 1.49 (0.70-3.20) | 1.49 (0.69-3.22) | 1.42 (1.12-1.80) |

Note, for Tables 6a and 6b, that fold changes in virulence gene expression are determined relative to the control condition through the 2^{^-ΔΔCt} method and are shown as the geometric mean (C.I.) (n = 3) of the 2^{^-ΔΔ} ^{Ct} values. All substrates are dissolved in PBS at a concentration of 1 M (upper part) or 1%(w/v) (lower part). Values between 0 and 1 represent relative downregulation, values >1 represent relative upregulation. Statistically significantly different fold changes relatively to the control (PBS) that are <0.5 (more than 2-fold downregulated) or >1.5 (more than 1.5-fold upregulated) are considered biologically relevant and are marked with ‘*’ (P < 0.05). NADG: N-acetyl-D-glucosamine; C.I.: 95% confidence interval.

In the case of A. actinomycetemcomitans, these downregulations ranged from 2-fold up to 100-fold (NADG), 2.4-fold to 100-fold (α-D-lactose), 2.5-fold to 100-fold (D-(+)-trehalose) and 5.9-fold to 100-fold (D-(+)-raffinose). Noteworthy is the significantly upregulated expression of pgA (11.6-fold for NADG, 4.3-fold for α-D-lactose, 18.2-fold for D-(+)-trehalose and 7.3-fold for D-(+)-raffinose). For P. gingivalis, downregulations ranged from 2.6-fold to 14-fold (NADG), 17-fold to 100-fold (α-D-lactose), 7.7-fold up to undetectable (D-(+)-trehalose) and 33-fold to 100-fold (D-(+)-raffinose).

In contrast, F. nucleatum virulence gene expression is in general significantly upregulated (2.5- to 250-fold) by all substrates relatively to the control in Table 6c at 1 M concentrations. Table 6c

Fold Change in virulence gene expression as measured by SYBR RT-qPCR**

| GENES | NADG 1 M | D-lactose 1 M | D-trehalose 1 M | D-raffinose 1 M |
|---|---|---|---|---|
| but. -coA transf. | 1.21 (0.99-1.49) | 3.88 (0.87-17.43)* | 4.28 (1.32-13.86)* | 3.95 (0.89-17.60)* |
| ompA | 1.33 (0.78-2.25) | 1.26 (0.79-2.03) | 1.23 (0.98-1.53) | 1.07 (0.49-2.36) |
| EF-G | 1.45 (0.93-2.26) | 1.54 (0.94-2.53) | 1.18 (0.46-3.02) | 1.41 (0.70-2.83) |
| ABC transp. perm. | 3.33 (2.53-4.39)^{∗} | 16.97 (5.93-48.61)^{∗} | 32.18 (21.08-49.14)^{∗} | 41.25 (11.65-146.10)^{∗} |
| transposase | 1.27 (0.68-2.37) | 172.87 (147.10-203.20)^{∗} | 106.44 (42.35-267.50)^{∗} | 251.47 (75.27-840.20)^{∗} |
| hemolysin | 1.90 (1.29-2.79) | 6.34 (1.36-29.47)^{∗} | 12.15 (7.73-19.10)^{∗} | 2.50 (1.07-5.83)^{∗} |
| hemin receptor | 3.26 (2.24-4.76)^{∗} | 36.87 (17.27-78.74)^{∗} | 41.19 (20.31-83.52)^{∗} | 18.04 (7.92-41.14)^{∗} |
| GENES | NADG 1% | D-lactose 1% | D-trehalose 1% | D-raffinose 1% |
| but. -coA transf. | 0.21 (0.11-0.39)^{∗} | 0.57 (0.40-0.83) | 0.40 (0.05-3.27) | 0.47 (0.06-3.98) |
| ompA | 0.74 (0.18-2.98) | 1.13 (0.58-2.19) | 0.84 (0.21-3.35) | 0.70 (0.20-2.44) |
| EF-G | 0.10 (0.02-0.45)^{∗} | 0.13 (0.03-0.51)^{∗} | 0.12 (0.03-0.55)^{∗} | 0.12 (0.04-0.33)^{∗} |
| ABC transp. perm. | 0.33 (0.12-0.87)^{∗} | 0.25 (0.08-0.78)^{∗} | 0.40 (0.15-1.03)^{∗} | 0.17 (0.04-0.65)^{∗} |
| transposase | 0.98 (0.18-5.39) | 1.26 (0.51-3.13) | 1.02 (0.33-3.12) | 0.78 (0.40-1.53) |
| hemolysin | 0.79 (0.30-2.04) | 1.00 (0.60-1.68) | 0.65 (0.15-2.70) | 0.51 (0.16-1.62)^{∗} |
| hemin receptor | 0.19 (0.02-1.67)^{∗} | 0.17 (0.03-0.94)^{∗} | 0.16 (0.05-0.50)^{∗} | 0.12 (0.09-0.15)^{∗} |

^{∗∗} Results are listed as fold changes relative to the control conditions (2^{^-ΔΔCt} method), the values for the control are normalized to “1”.

Note, for Table 6c, fold changes in virulence gene expression are determined relative to the control condition through the 2^{^-ΔΔCt} method and are shown as the geometric mean (C.I.) (n = 3) of the 2^{^-ΔΔCt} values. All substrates are dissolved in PBS at a concentration of 1 M (upper part) or 1%(w/v) (lower part). Values between 0 and 1 represent relative downregulation, values >1 represent relative upregulation. Statistically significantly different fold changes relatively to the control (PBS) that are <0.5 (more than 2-fold downregulated) or >1.5 (more than 1.5-fold upregulated) are considered biologically relevant and are shown in bold and marked with ‘*’ (P < 0.05). NADG: N-acetyl-D-glucosamine; C.I.: 95% confidence interval.

However, the upregulation induced by NADG at 1 M is more limited and only significant for 2 genes, encoding the ABC transporter permease and the hemin receptor.

For P. intermedia, there is a more diverse impact of the substrates on virulence gene expression and the results are demonstrated in Table 6d: Table 6d:

Fold Change in virulence gene expression as measured by SYBR RT-qPCR^{∗∗}

| Genes | NADG 1 M | D-lactose 1 M | D-trehalose 1 M | D-raffinose 1 M |
|---|---|---|---|---|
| adpc | 2.65 (1.25-5.62)* | 0.30 (0.23-0.39)^{∗} | 1.45 (0.76-2.77) | 1.02 (0.40-2.60) |
| clpB | 2.03 (0.94-4.41) | 0.38 (0.08-1.75)^{∗} | 1.90 (0.77-4.68) | 2.21 (1.53-3.21) |
| DnaK | 2.34 (1.23-4.43)* | 0.34 (0.28-0.42)^{∗} | 8.82 (6.83-11.38)^{∗} | 10.07 (6.81-14.89)^{∗} |
| DnaJ | 0.64 (0.35-1.16) | 0.25 (0.05-1.25)^{∗} | 1.81 (1.21-2.71) | 1.76 (0.92-3.37) |
| ECF | 20.32 (4.81-85.88)^{∗} | 1.72 (0.56-5.25) | 6.97 (1.65-29.40)^{∗} | 6.74 (1.96-23.20)^{∗} |
| GroES | 1.86 (1.38-2.50) | 0.29 (0.09-0.91)^{∗} | 11.52 (2.46-53.90)^{∗} | 10.59 (4.96-22.60)^{∗} |
| HtpG | 3.40 (2.27-5.08)* | 0.19 (0.12-0.31)^{∗} | 0.67 (0.43-1.03) | 1.90 (0.99-3.67)^{∗} |
| KpsD | 1.72 (0.62-4.72) | 0.62 (0.21-1.87) | 0.59 (0.47-0.75) | 1.78 (0.71-4.45) |
| inpA | 4.52 (4.14-4.94)^{∗} | 0.47 (0.27-0.82)^{∗} | 0.81 (0.41-1.63) | 1.39 (0.67-2.90) |
| phg | 0.57 (0.38-0.85)* | 0.12 (0.08-0.18)^{∗} | 0.20 (0.14-0.29)^{∗} | 0.33 (0.21-0.50)^{∗} |
| Genes | NADG 1 M | D-lactose 1 M | D-trehalose 1 M | D-raffinose 1 M |
| adpc | 0.67 (0.64-0.69) | 0.41 (0.30-0.55) | 0.64 (0.29-1.4) | 0.85 (0.23-3.13) |
| clpB | 1.06 (0.58-1.96) | 0.85 (0.23-3.13) | 1.30 (0.44-3.83) | 1.13 (0.86-1.48) |
| DnaK | 0.77 (0.34-1.77) | 1.16 (0.55-2.44) | 0.78 (0.44-1.36) | 1.76 (0.61-5.06) |
| DnaJ | 0.70 (0.50-0.99) | 0.29 (0.18-0.47)^{∗} | 0.62 (0.08-4.67) | 1.56 (0.59-4.12) |
| ECF | 0.48 (0.11-2.02) | 0.26 (0.12-0.58)^{∗} | 0.69 (0.53-0.91) | 0.96 (0.56-1.64) |
| GroES | 0.75 (0.49-1.14) | 0.91 (0.41-2.03) | 1.22 (0.61-2.45) | 0.98 (0.24-4.05) |
| HtpG | 0.46 (0.22-0.94)* | 0.40 (0.15-1.07)^{∗} | 0.69 (0.42-1.12) | 0.67 (0.43-1.04) |
| KpsD | 0.78 (0.37-1.62) | 0.72 (0.39-1.35) | 1.26 (0.52-3.08) | 0.90 (0.58-1.40) |
| inpA | 0.53 (0.24-1.17)* | 0.52 (0.41-0.66)^{∗} | 0.56 (0.31-1.01)^{∗} | 0.98 (0.64-1.52) |
| phg | 0.69 (0.47-1.00) | 0.39 (0.19-0.77)^{∗} | 0.86 (0.82-0.89) | 0.73 (0.33-1.63) |

^{∗∗} Results are listed as fold changes relative to the control conditions (2^{^-ΔΔCt} method), the values for the control are normalized to “1”.

Note, for Table 6d, fold changes in virulence gene expression are determined relatively to the control condition through the 2^{^-ΔΔCt} method and are shown as the geometric mean (C.I.) (n = 3) of the 2^{^-ΔΔCt} values. All substrates are dissolved in PBS at a concentration of 1 M (upper part) or 1%(w/v) (lower part). Values between 0 and 1 represent relative downregulation, values >1 represent relative upregulation. Statistically significantly different fold changes relatively to the control (PBS) that are <0.5 (more than 2-fold downregulated) or >1.5 (more than 1.5-fold upregulated) are considered biologically relevant and are shown in bold and marked with ‘*’ (P < 0.05). NADG: N-acetyl-D-glucosamine; C.I.: 95% confidence interval.

In Table 6d, there is a more diverse impact of the substrates on virulence gene expression. For example, α-D-lactose significantly downregulated the expression of various virulence genes (approximately 2.1- to 8.3-fold), By contrast, NADG (approximately 2.3- to 20.3-fold upregulation), D-(+)-trehalose (7- to 11.5-fold upregulation and 5-fold downregulation) and D-(+)-raffinose (1.9- to 10.6-fold upregulation and 3-fold downregulation) significantly up- or downregulated them.

In multi-species biofilms rinsed with the substrates at concentrations of 1% (_{w/v}), there is generally a significantly decreased A. actinomycetemcomtans virulence gene expression (2.3- to 25-fold) as demonstrated in Table 6a above. Only for the NADG condition, the expression of one gene (orf859) is significantly upregulated (2-fold). The substrates, at concentrations of 1% _{(w/v)}, appear to have a limited impact on P. gingivalis virulence gene expression, with only D-(+)-trehalose and NADG having a significant impact on kgp (approximately 3.2-fold up-regulation) and partC (approximately 2.8-fold downregulation) expression, respectively, as demonstrated in Table 6b above. For F. nucleatum, and in contrast with the results obtained for substrate concentrations of 1 M, there is significantly decreased virulence gene expression (approximately 2- to 10-fold) for most of the substrates as demonstrated in Table 6c above. For P. intermedia, D-(+)-trehalose and D-(+)-raffinose only had a limited impact on virulence gene expression, whereas NADG and α-D-lactose in general significantly downregulated virulence gene expression (approximately 2.1- to 3.8-fold) as demonstrated in Table 6d above.

### Example 4 - Effects of Substrates on Multi-Species Biofilm Inflammatory Potential

Cultures of immortalized human oral keratinocytes (HOK-18A) are grown. The relative inflammatory potential of the substrate-treated multi-species biofilms is evaluated by analyzing the expression of five inflammatory mediator genes in human oral keratinocytes (HOKs) exposed to the substrate-treated biofilms. significantly different gene expressions in HOKs exposed the substrate-treated biofilms relatively to HOKs exposed to the control biofilms are considered to be biologically relevant if their value is more than 1.5-fold upregulated or more than 2-fold downregulated. Only these results are considered. The IL-8 levels in the cellular supernatant are determined as well. RNA is converted to cDNA and relative expression of inflammatory mediator genes is determined as described above with respect to the cellular housekeeping gene β-actin.

In HOKs exposed to substrate-treated (substrate concentrations of 1 M) multi-species biofilms, there are mostly decreases in inflammatory mediator gene expression as measured via SYBR RT-qPCR, the results of which are detailed in Table 7:

TABLE 7

- Effects of repeated rinsing with potentially prebiotic substrates on multi-species biofilm inflammatory potential towards human oral keratinocytes.

| Genes | NADG 1 M | D-lactose 1 M | D-trehalose 1 M | D-raffinose 1 M |
|---|---|---|---|---|
| IL-1β | 0.75 (0.55-1.02) | 0.5 (0.38-0.65)^{∗} | 0.79 (0.45-1.38) | 0.8 (0.44-1.46) |
| IL-6 | 0.57 (0.46-0.71) | 0.33 (0.12-0.88)^{∗} | 1.27 (0.11-14.86) | 0.54 (0.17-1.72) |
| IL-8 | 0.14 (0.06-0.36)^{∗} | 0.03 (0.02-0.05)^{∗} | 0.22 (0.15-0.34)^{∗} | 0.10 (0.04-0.29)^{∗} |
| MMP-8 | 1.28 (0.79-2.08) | 0.78 (0.38-1.61) | 0.81 (0.68-0.95) | 0.85 (0.61-1.19) |
| TNF-α | 0.89 (0.59-1.34) | 0.39 (0.23-0.66)^{∗} | 0.66 (0.41-1.06)^{∗} | 0.66 (0.56-0.78)^{∗} |
| Genes | NADG 1% | D-lactose 1% | D-trehalose 1% | D-raffinose 1% |
| IL-1β | 0.71 (0.38-1.33)^{∗} | 0.89 (0.54-1.45) | 1.06 (0.55-2.06) | 0.93 (0.56-1.55) |
| IL-6 | 0.73 (0.58-0.93)^{∗} | 0.74 (0.56-0.97)^{∗} | 1.00 (0.46-2.17) | 0.70 (0.44-1.13)^{∗} |
| IL-8 | 1.00 (0.78-1.27) | 1.13 (0.71-1.80) | 1.32 (0.89-1.94)^{∗} | 1.08 (0.73-1.59) |
| MMP-8 | 0.52 (0.17-1.6)^{∗} | 0.70 (0.62-0.79) | 0.66 (0.46-0.93) | 0.71 (0.38-1.35) |
| TNF-α | 0.71 (0.28-1.35)^{∗} | 0.81 (0.50-1.33) | 0.94 (0.54-1.64) | 0.77 (0.55-1.08) |

Fold changes in inflammatory mediator gene expression from human oral keratinocytes (HOK-18A) exposed to substrate-treated multi-species biofilms are determined relatively to the control through the 2^{^-ΔΔCt} method and are shown as the geometric mean (C.I.) (n = 3) of the 2^{^-ΔΔCt} values. All substrates are dissolved in PBS at a concentration of 1 M (upper part) or 1%(w/v) (lower part). Values between 0 and 1 represent relative downregulation, values >1 represent relative upregulation. Statistically significantly different fold changes relatively to the control (PBS) that are <0.5 (more than 2-fold downregulated) or >1.5 (more than 1.5-fold upregulated) are considered biologically relevant and are shown in bold and marked with ‘*’ (P < 0.05).

IL-8 gene expression (pg/mL) is significantly downregulated for all substrates ranging from 4.5-fold to 33.3-fold. TNF-α gene expression is significantly downregulated for the α-D-lactose condition (2.5-fold) and this is also the case for IL-1β gene expression (2-fold) and IL-6 gene expression (3-fold). Absolute IL-8 levels (pg/mL) are significantly reduced for all substrate conditions, ranging from 8.4-fold to undetectable. This data is demonstrated in Table 8:

TABLE 8

- Effects of repeated rinsing with potentially prebiotic substrates on multi-species biofilm inflammatory potential
Mean ± SD IL-8 levels (mg/L)

| 1 M | control | NADG | D-lactose | D-trehalose | D-raffinose |
|---|---|---|---|---|---|
| | 21.43 ± 4.67 | 0.22 ± 0.32^{∗} | < LOD^{∗} | 2.56 ± 0.97^{∗} | 1.47 ± 1.72^{∗} |
| 1% (w/v) | control | NADG | D-lactose | D-trehalose | D-raffinose |
| | 72.52 ± 19.20 | 54.75 ± 11.66 | 53.42 ± 5.22 | 93.45 ± 18.35 | 74.19 ± 5.29 |

IL-8 levels detected in the supernatants of human oral keratinocytes (HOK-18A) cultures exposed to substrate-treated multi-species biofilms are shown as mean ± SD (n = 3) values (pg/mL). Substrates are dissolved in PBS at a concentration of 1 M (upper row) or 1% (w/v) (lower row).
Statistically significantly different values in comparison to the control (PBS) are marked with ‘^{∗}’ (P < 0.05). IL-8: interleukin-8; NADG: N-acetyl-D-glucosamine; LOD: limit of detection.

As demonstrated in Table 7, in HOKs exposed to substrate-treated (substrate concentrations of 1%_{(w/v)}) multi-species biofilms, the relative expression of most inflammatory mediator genes is generally unaffected. This is also consistent with the observations for IL-8 gene expression noted above, where absolute IL-8 levels are not significantly affected.

### Example 5 - Compositions Comprising Saccharide Prebiotic

A toothpaste comprising a saccharide prebiotic, e.g., N-acetyl-D-glucosamine, α-D-lactose, D-(+)-trehalose or D-(+)-raffinose, is potentially prepared using the following ingredients:

| Ingredient | % |
|---|---|
| 70% Sorbitol | 20 |
| Glycerin | 20 |
| Water | Q.S. |
| High Cleaning Silica | 10 |
| Gantrez S-97 | 15 |
| Abrasive Silica | 8.8 |
| Thickening Silica | 2.7 |
| Sodium Lauryl Sulfate | 1.5 |
| Sodium Hydroxide | 0 - 1.2 |
| Sodium CMC - Type 12 | 1.1 |
| Flavor | 1 - 1.2 |
| Titanium Dioxide | 0.75 |
| Propylene Glycol | 0.5 |
| Carrageenan Gum | 0.48 |
| Sodium Saccharin | 0.3 |
| Saccharide prebiotic (e.g., N-acetyl-D-glucosamine, α-D-lactose, | |
| D-(+)-trehalose or D-(+)-raffinose) | 1 |
| Total | 100% |

Another toothpaste comprising a saccharide prebiotic is potentially prepared using the following ingredients:

| Ingredient | % |
|---|---|
| 70% Sorbitol | 14 |
| Glycerin | 17 |
| Water | Q.S. |
| High Cleaning Silica | 17 |
| Gantrez S-97 | 17 |
| Thickening Silica | 2.7 |
| Sodium Lauryl Sulfate | 1.5 |
| Sodium Hydroxide | 0 - 1.2 |
| Sodium CMC - Type 12 | 1.1 |
| Xanthan Gum | 0.8 |
| Flavor | 1-1.2 |
| Titanium Dioxide | 0.5 |
| Propylene Glycol | 0.5 |
| Carrageenan Gum | 0.48 |
| Sodium Saccharin | 0.3 |
| Sodium Fluoride | 0.243 |
| Saccharide prebiotic (e.g., N-acetyl-D-glucosamine, α-D-lactose, D-(+)-trehalose or D-(+)-raffinose) | 0.5 |
| Total | 100% |

1. An oral care composition comprising an effective amount of at least one saccharide prebiotic selected from N-acetyl-D-glucosamine, α-D-lactose, D-(+)-trehalose or D-(+)-raffinose and mixtures thereof in an amount effective to promote the growth of beneficial endogenous bacteria in the oral cavity and inhibit pathogenic oral bacteria.

2. The oral care composition of claim 1, wherein the saccharide prebiotic is N-acetyl-D-glucosamine.

3. The oral care composition of claim 1, wherein the saccharide prebiotic is α-D-lactose.

4. The oral care composition of claim 1, wherein the saccharide prebiotic is D-(+)-trehalose.

5. The oral care composition of claim 1, wherein the saccharide prebiotic is D-(+)-raffinose.

6. (canceled)

7. (canceled)

8. The oral care composition of claim 1, wherein the composition promotes the growth or expression in the oral cavity of one or more beneficial endogenous bacterial species, wherein said species are one or more selected from the group consisting of: A. naeslundii; A. viscosus; S. gordonii: S. mitis; S. oralis; S. salivarius; S. sanguinis; V. parvula.

9. The oral care composition of claim 1, wherein the composition negatively affects the growth or expression in the oral cavity of one or more pathogenic bacterial species, wherein said species are one or more selected from the group consisting of: A. actinomycetemcomitans; F. nucleatum; P. gingivalis; P. intermedia; S. mutans; S. Sobrinus.

10. (canceled)

11. The oral care composition of claim 1, wherein the composition further comprises at least one species of bacteria that has beneficial effects on oral health.

12. The oral care composition of claim 11, wherein the species of bacteria that has beneficial effects on oral health is selected from A. naeslundii; A. viscosus; S. gordonii: S. mitis; S. oralis; S. salivarius; S. sanguinis; V. parvula and combinations thereof.

13. The oral care composition of claim 1, wherein the composition is in the form selected from: a mouthwash, toothpaste, tooth gel, tooth powder, non-abrasive gel, mousse, foam, mouth spray, lozenge, oral tablet, and dental implement.

14. (canceled)

15. The oral care composition of claim 1, wherein the prebiotic substrate can decrease virulence gene expression is selected from one or more of the following genes in A. actinomycetemcomitans: flp, aae, apaH, cdtB, emaA, ltxA, omp100, omp29, orf859, pgA, vapA, and vppA.

16. The oral care composition of claim 1, wherein the prebiotic substrate can decrease virulence gene expression is selected from one or more of the following genes in P. gingivalis: fim A, kgp, partC, and rgpA.

17. The oral care composition of claim 1, wherein the prebiotic substrate can decrease virulence gene expression is selected from one or more of the following genes in P. intermedia: adpc, clpB, DnaK, DnaJ, ECF, GroES, HtpG, KpsD, inpA, and phg.

18. The oral care composition of claim 1, wherein the prebiotic substrate can decrease virulence gene expression is selected from one or more of the following genes in F. nucleatum: butyrate-acetoacetate CoA-transferase., ABC transporter permease, transposase, hemolysin, hemin receptor, ompA, and EF-G.

19. The oral care composition of claim 1, wherein the composition is in an amount sufficient to decrease the gene expression of one or more inflammatory biomarker in oral keratinocytes.

20. The composition of claim 19, wherein the biomarker is selected from: IL-1β, IL-6, MMP-8, TNF-α, IL-8, and combinations thereof.

21. A method for selectively promoting, in an oral cavity: growth, metabolic activity or colonization of bacteria that have beneficial effects on oral health, relative to growth, metabolic activity or colonization of pathogenic oral bacteria, comprising administering to the oral cavity an oral care composition comprising a composition of claim 1 .

22. A method for selectively promoting, in an oral cavity, biofilm formation by bacteria that have beneficial effects on oral health, relative to biofilm formation by pathogenic oral bacteria, comprising administering to the oral cavity an oral care composition comprising a composition of claim 1 .

23. A method for preventing or mitigating one or more of gingivitis, periodontitis, peri-implantitis, peri-implant mucositis, necrotizing gingivitis, necrotizing periodontitis and caries in a subject in need thereof, by selectively promoting in the oral cavity of the subject the growth, metabolic activity or colonization of bacteria that have beneficial effects on oral health, relative to growth, metabolic activity or colonization of pathogenic oral bacteria, comprising administering to the oral cavity an oral care composition comprising a composition of claim 1 .

24. (canceled)

25-34. (canceled)
